(19) United States
(12) Patent Application Publication
(10) Pub. No.: US 2007/0004797 A1
(43) Pub. Date: Jan. 4, 2007

(54) Methods and dosage forms for reducing side effects of carbamate compounds

(76) Inventors: Richard G. Weyers, Los Altos, CA (US); Gayatri Sathyan, San Jose, CA (US); Ying Ou, Sunnyvale, CA (US); Suneel K. Gupta, Sunnyvale, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/478,241

(22) Filed: Jun. 29, 2006

Related U.S. Application Data
(60) Provisional application No. 60/695,715, filed on Jun. 29, 2005.

(51) Int. Cl. A61K 31/325

(52) U.S. Cl. 514/483; 514/489

(57) ABSTRACT

Disclosed are dosage forms and methods comprising compounds of Formula (I) and Formula (II). More particularly, disclosed are dosage forms, methods, and new uses of compounds of Formula (I) and Formula (II) that substantially reduce or substantially eliminate certain side effects of the compounds of Formula (I) and Formula (II) when dosed to a patient.

### CROSS REFERENCE TO RELATED U.S. APPLICATION INFO

The present application claims the benefit under 35 USC §119(e) of U.S. Provisional application 60/695,715 filed Jun. 29, 2005.

### FIELD OF THE INVENTION

The invention relates to dosage forms and methods comprising carbamate compounds. More particularly, the invention relates to dosage forms, methods, and new uses of carbamate compounds that substantially reduce or substantially eliminate certain side effects of the carbamate compounds when dosed to a patient.

### BACKGROUND

Substituted phenyl alkyl carbamate compounds have been described in U.S. Pat. No. 3,265,728 to Bossinger, et al (incorporated herein by reference) as useful in treating the central nervous system, having tranquilization, sedation and muscle relaxation properties of the formula:

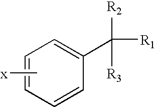

wherein R₁ is either carbamate or alkyl carbamate containing from 1 to 3 carbon atoms in the alkyl group; R₂ is either hydrogen, hydroxy, alkyl or hydroxy alkyl containing from 1 to 2 carbons; R₃ is either hydrogen or alkyl containing from 1 to 2 carbons; and X can be halogen, methyl, methoxy, phenyl, nitro or amino.

A method for inducing calming and muscle relaxation with carbamates has been described in U.S. Pat. No. 3,313,692 to Bossinger, et al (incorporated herein by reference) by administering a compound of the formula:

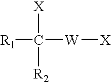

in which W represents an aliphatic radical containing less than 4 carbon atoms, wherein R₁ represents an aromatic radical, R₂ represents hydrogen or an alkyl radical containing less than 4 carbon atoms, and X represents a hydrogen, hydroxy, alkoxy or alkyl radical containing less than 4 carbon atoms or a radical of the formula:

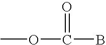

in which B represents an organic heterocyclic, ureido or hydrazino amine radical or the radical —N(R₃)₂, wherein R₃ represents hydrogen or an alkyl radical containing less than 4 carbon atoms.

Optically pure forms of substituted phenyl alkyl carbamate compounds have been described in U.S. Pat. No. 6,103,759 to Choi, et al (incorporated herein by reference) as effective for treating and preventing central nervous system disorders including convulsions, epilepsy, stroke and muscle spasm and as useful in the treatment of central nervous system diseases, particularly as anticonvulsants, antiepileptics, neuroprotective agents and centrally acting muscle relaxants and, in particular, as halogen substituted 2-phenyl-1,2-ethanediol monocarbamate and dicarbamate compounds of the formulae:

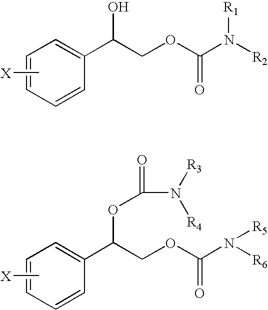

wherein one enantiomer predominates and wherein the phenyl ring is substituted at X with one to five halogen atoms selected from fluorine, chlorine, bromine or iodine atoms and R₁, R₂, R₃, R₄, R₅ and R₆ are each selected from hydrogen and straight or branched alkyl groups with one to four carbons optionally substituted with a phenyl group with substituents selected from the group consisting of hydrogen, halogen, alkyl, alkyloxy, amino, nitro and cyano. Pure enantiomeric forms and enantiomeric mixtures are described wherein one of the enantiomers predominates in the mixture for the compounds represented by the formulae above; preferably, one of the enantiomers predominates to the extent of about 90% or greater; and, most preferably, about 98% or greater.

Administration of certain carbamate compounds may lead to dose-dependent side effects, including but not limited to dizziness and sedation. Accordingly, there is a need for effective dosing methods, dosage forms and devices that will permit the dosing of such carbamate compounds in a way that reduces side effects. Exemplary methodologies, dosage forms, methods of preparing such dosage forms and methods of using such dosage forms are disclosed herein.

### BRIEF DESCRIPTION OF THE DRAWINGS

FIG. 1 shows an oral dosage form according to the invention.

FIG. 2 shows an oral dosage form according to the invention.

FIG. 3 shows an oral dosage form according to the invention.

FIG. 4 shows an oral dosage form according to the invention.

FIGS. 5A-C show an oral dosage form according to the invention.

FIG. 6 shows an oral dosage form according to the invention.

FIG. 7 shows plasma concentration curves.

### DETAILED DESCRIPTION

Introduction

The inventors have unexpectedly discovered that the aforementioned problems can be addressed by providing oral dosage forms and methods that comprise a dose of a compound of Formula (I) or Formula (II) and dosing structures or sustainable releasing means that provide specified release profiles or, following dosing, specified pharmacokinetic characteristics. In particular, the inventors have noted that a significant reduction in side effects can be achieved by practicing the present invention.

The invention will now be described in more detail below.

Definitions

All percentages are weight percent unless otherwise noted.

All references cited herein are incorporated herein by reference in their entirety and for all purposes to the same extent as if each individual publication or patent or patent application was specifically and individually indicated to be incorporated by reference in its entirety for all purposes. The discussion of references herein is intended merely to summarize the assertions made by their authors and no admission is made that any reference constitutes prior art. Applicants reserve the right to challenge the accuracy and pertinence of the cited references.

The present invention is best understood by reference to the following definitions, the drawings and exemplary disclosure provided herein.

“Administering” or “administration” means providing a drug to a patient in a manner that is pharmacologically useful.

“Apparent terminal half-life” (t_{1/2}) is calculated as 0.693/k, wherein “k” means the apparent elimination rate constant, estimated by linear regression of the log-transformed plasma concentration during the terminal log-linear elimination phase.

“Area under the curve” or “AUC” is the area as measured under a plasma drug concentration curve. Often, the AUC is specified in terms of the time interval across which the plasma drug concentration curve is being integrated, for instance AUC_{start-finish}. Thus, AUC₀₋₄₈ refers to the AUC obtained from integrating the plasma concentration curve over a period of zero to 48 hours, where zero is conventionally the time of administration of the drug or dosage form comprising the drug to a patient. AUCₜ refers to area under the plasma concentration curve from hour 0 to the last detectable concentration at time t, calculated by the trapezoidal rule. AUC_{inf} refers to the AUC value extrapolated to infinity, calculated as the sum of AUCₜ and the area extrapolated to infinity, calculated by the concentration at time t (Ct) divided by k. (If the t_{1/2} value was not estimable for a subject, the mean t_{1/2} value of that treatment was used to calculate AUC_{inf}.). “Mean, single dose, area under a plasma concentration-time curve AUC_{inf}” means the mean AUC_{inf} obtained over several patients or multiple administrations to the same patient on different occasions with sufficient washout in between dosings to allow drug levels to subside to pre-dose levels, etc., following a single administration of a dosage form to each patient.

“Ascending plasma concentration” means a drug plasma concentration profile over about the first 12 to 24 hours following initial dosing, wherein the profile shows an increase to a maximum concentration, wherein said maximum occurs more than about 9 hours following the initial dose, preferably, more than about 10 hours following initial dose, more preferably, more than about 12 hours after dose.

Persons of skill in the art will appreciate that blood plasma drug concentrations obtained in individual subjects will vary due to interpatient variability in the many parameters affecting drug absorption, distribution, metabolism and excretion. For this reason, unless otherwise indicated, when a drug plasma concentration is listed, the value listed is the calculated mean value based on values obtained from a groups of subjects tested.

“Ascending rate of release” or “ascending release rate” means a rate of release wherein the amount of drug released from a dosage form as a function of time increases over a period of time, preferably continuously and gradually. Preferably, the rate of drug released as a function of time increases in a steady (rather than step-wise) manner. More preferably, an ascending rate of release may be characterized as follows. The rate of release as a function of time for a dosage form is measured and plotted as % drug release versus time or as milligrams of drug released/hour versus time. An ascending rate of release is preferably characterized by an average rate (expressed in mg of drug per hour) wherein the rate within a given two hour span is higher as compared with the previous two hour time span, over the period of time of about 2 hours to about 12 hours, preferably, about 2 hours to about 18 hours, more preferably about 4 hours to about 12 hours, more preferably still, about 4 hours to about 18 hours. Preferably, the increase in average rate is gradual such that less than about 30% of the dose is delivered during any 2 hour interval, more preferably, less than about 25% of the dose is delivered during any 2 hour interval. Preferably, the ascending release rate is maintained until at least about 50%, more preferably until at least about 75% of the drug in the dosage form has been released.

In other preferably embodiments, ascending rates of release may be defined with reference to specific release rates measured at specified times following administration of the dosage form in question. Preferably such release rates are determined in vitro.

“C” means the concentration of drug in blood plasma, or serum, of a subject, generally expressed as mass per unit volume, typically nanograms per milliliter. For convenience, this concentration may be referred to herein as “drug plasma concentration”, “plasma drug concentration” or “plasma concentration”. The plasma drug concentration at any time following drug administration is referenced as Ctime, as in C9 h or C24 h, etc. A maximum plasma concentration obtained following administration of a dosage form obtained directly from the experimental data without interpolation is referred to as Cmax. The average or mean plasma concentration obtained during a period of interest is referred to as Cavg or Cmean. “Mean, single dose, maximum plasma concentration Cmax” means the mean Cmax obtained over several patients or multiple administrations to the same patient with sufficient washout in between dosings to allow drug levels to subside to pre-dose levels, etc., etc., following a single administration of a dosage form to each patient.

“Composition” means a product containing a compound of the present invention (such as a product comprising the specified ingredients in the specified amounts, as well as any product which results, directly or indirectly, from such combinations of the specified ingredients in the specified amounts).

“Compound” or “drug” means a compound of

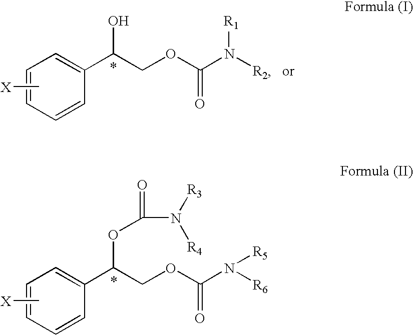

or pharmaceutically acceptable forms thereof, wherein phenyl is substituted at X with one to five halogen atoms selected from the group consisting of fluorine, chlorine, bromine and iodine, and R₁, R₂, R₃, R₄, R₅ and R₆ are independently selected from the group consisting of hydrogen and C₁-C₄ alkyl, wherein C₁-C₄ alkyl is optionally substituted with phenyl and, wherein phenyl is optionally substituted with substituents independently selected from the group consisting of halogen, C₁-C₄ alkyl, C₁-C₄ alkoxy, amino, nitro and cyano.

Where other crystalline or polymorphic forms of the instant compounds may exist, as such they are also intended to be included within the scope of the present invention. Compounds of the present invention may be prepared as described generally in U.S. Pat. No. 3,265,728 to Bossinger et al. (“Bossinger '728”), U.S. Pat. No. 3,313,692 to Bossinger et al. (“Bossinger '692”) and U.S. Pat. No. 6,103,759 to Choi et al. (“Choi '759”). It is understood that substituents and substitution patterns on the compounds of this invention can be selected by one of ordinary skill in the art to provide compounds that are chemically stable and that can be readily synthesized by techniques known in the art as well as those methods set forth herein.

In embodiments, the compound comprises an enantiomer selected from Formula (Ia) or Formula (IIa), a racemic mixture of Formula (Ia) or Formula (IIa), or an enantiomeric mixture of Formula (Ia) or Formula (IIa) wherein one enantiomer predominates:

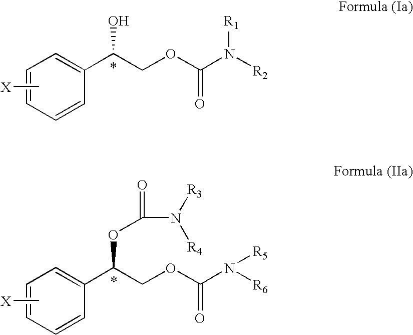

or pharmaceutically acceptable forms thereof, wherein phenyl is substituted at X with one to five halogen atoms selected from the group consisting of fluorine, chlorine, bromine and iodine, and R₁, R₂, R₃, R₄, R₅ and R₆ are independently selected from the group consisting of hydrogen and C₁-C₄ alkyl, wherein C₁-C₄ alkyl is optionally substituted with phenyl and, wherein phenyl is optionally substituted with substituents independently selected from the group consisting of halogen, C₁-C₄ alkyl, C₁-C₄ alkoxy, amino, nitro and cyano.

In another embodiment, the compound is selected from from Formula (Ia) or Formula (IIa), wherein X is chlorine and, wherein X is substituted at the ortho position of the phenyl ring. In another embodiment, an enantiomer is selected from Formula (Ia) or Formula (IIa) or enantiomeric mixture thereof wherein one enantiomer predominates and, wherein R₁, R₂, R₃, R₄, R₅ and R₆ are hydrogen.

In an embodiment, for enantiomeric mixtures wherein one enantiomer selected from Formula (Ia) or Formula (IIa) predominates, said enantiomer predominates to the extent of about 90% or greater. Enantiomeric mixtures within the scope of the present invention include those wherein said enantiomer predominates to the extent of about 98% or greater.

In embodiments, the compound comprises an enantiomer selected from Formula (Ib) or Formula (IIb), a racemic mixture of Formula (Ia) or Formula (IIa), or an enantiomeric mixture of Formula (Ib) or Formula (IIb) wherein one enantiomer predominates:

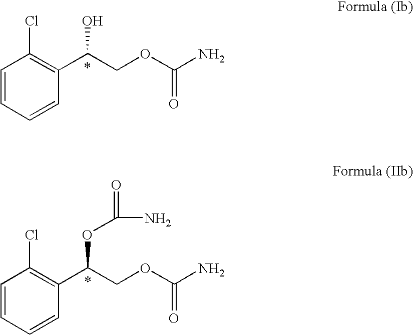

It is apparent to those skilled in the art that the compounds of the invention may be present as racemates, enantiomers and enantiomeric mixtures thereof. A carbamate enantiomer selected from Formula (I), Formula (II), Formula (Ia), Formula (IIa), Formula (Ib) or Formula (IIb) contains an asymmetric chiral carbon atom at the benzylic position, which is the aliphatic carbon adjacent to the phenyl ring (represented by the asterisk in the structural formulae).

Examples of a compound selected from Formula (I) for use in the present invention include an enantiomer of Formula (I) or an enantiomer of Formula (I) in an enantiomeric mixture wherein one enantiomer predominates.

Examples of a compound selected from Formula (II) for use in the present invention include an enantiomer of Formula (II) or an enantiomer of Formula (II) in an enantiomeric mixture wherein one enantiomer predominates.

For an enantiomeric mixture of Formula (I) or Formula (II) wherein one enantiomer predominates, the enantiomer preferably predominates to the extent of about 90% or greater. Examples of the present invention also include enantiomeric mixtures wherein said enantiomer preferably predominates to the extent of about 98% or greater.

Other examples of said compound of Formula (I) or Formula (II) include compounds wherein X is chlorine, wherein X is substituted at the ortho position of the phenyl ring of Formula (I) or Formula (II) and, wherein R₁, R₂, R₃, R₄, R₅ and R₆ are hydrogen.

An example of the present invention includes the use of an enantiomer selected from Formula (I) or Formula (II) or an enantiomeric mixture thereof wherein one enantiomer predominates, wherein X is chlorine and, wherein X is substituted at the ortho position of the phenyl ring.

The present invention also includes the use of an enantiomer selected from Formula (I) or Formula (II) or an enantiomeric mixture thereof wherein one enantiomer predominates and, wherein R₁, R₂, R₃, R₄, R₅ and R₆ are hydrogen. In an embodiment, for enantiomeric mixtures wherein one enantiomer selected from Formula (I) or Formula (II) predominates, said enantiomer preferably predominates to the extent of about 90% or greater. Enantiomeric mixtures within the scope of the present invention also include those wherein said enantiomer preferably predominates to the extent of about 98% or greater.

Examples of the present invention include the use of an enantiomer selected from Formula (Ia) or Formula (IIa) or an enantiomeric mixture thereof wherein one enantiomer predominates, wherein X is chlorine and, wherein X is substituted at the ortho position of the phenyl ring.

The present invention also includes the use of an enantiomer selected from Formula (Ia) or Formula (IIa) or enantiomeric mixtures thereof wherein one enantiomer predominates and, wherein R₁, R₂, R₃, R₄, R₅ and R₆ are hydrogen.

For enantiomeric mixtures wherein one enantiomer selected from Formula (Ia) or Formula (IIa) predominates, said enantiomer preferably predominates to the extent of about 90% or greater. Enantiomeric mixtures within the scope of the present invention include those wherein said enantiomer preferably predominates to the extent of about 98% or greater.

Examples of the present invention include the use of an enantiomer selected from Formula (Ib) or Formula (IIb) or an enantiomeric mixture thereof wherein one enantiomer predominates, wherein X is chlorine and, wherein X is substituted at the ortho position of the phenyl ring.

The present invention also includes the use of an enantiomer selected from Formula (Ib) or Formula (IIb) or enantiomeric mixtures thereof wherein one enantiomer predominates and, wherein R₁, R₂, R₃, R₄, R₅ and R₆ are hydrogen.

For enantiomeric mixtures wherein one enantiomer selected from Formula (Ib) or Formula (IIb) predominates, said enantiomer preferably predominates to the extent of about 90% or greater. Enantiomeric mixtures within the scope of the present invention include those wherein said enantiomer preferably predominates to the extent of about 98% or greater.

“Dosage form” means one or more compounds in a medium, carrier, vehicle, or device suitable for administration to a patient. “Oral dosage form” means a dosage form suitable for oral administration.

“Dose” means a unit of drug. Conventionally, a dose is provided as a dosage form. Doses may be administered to patients according to a variety of dosing regimens. Common dosing regimens include once daily orally (qd), twice daily orally (bid), and thrice daily orally (tid).

“Effective amount” means that amount of compound that elicits the biological or medicinal response in a tissue system, animal or human, that is being sought by a researcher, veterinarian, medical doctor, or other clinician, which includes therapeutic alleviation of the symptoms of the disease or disorder being treated and prophylactic. The effective amount of a compound selected from Formula (I) or Formula (II) or pharmaceutical composition thereof may be from about 0.01 mg/Kg/dose to about 300 mg/Kg/dose. Effective amounts may also be from about 0.01 mg/Kg/dose to about 100 mg/Kg/dose. An effective amount also contemplated may be from about 0.05 mg/Kg/dose to about 10 mg/Kg/dose. Another effective amount includes from about 0.1 mg/Kg/dose to about 5 mg/Kg/dose. Therefore, the effective amount of the active ingredient contained per dosage unit as described herein may be in a range of from about 700 ng/dose to about 21 g/dose for a subject having a weight of about 70 Kg.

“Enantiomer” means one of a pair of molecular species that are mirror images of each other and are not superposable. The term “diastereomer” refers to stereoisomers that are not related as mirror images. The symbols “R” and “S” represent the configuration of substituents around a chiral carbon atom(s). The symbols “R*” and “S*” denote the relative configurations of of substituents around a chiral carbon atom(s). The isomeric descriptors “R,” “S,” “S*” or “R*” are used as described herein for indicating atom configuration(s) relative to a core molecule and are intended to be used as defined in the literature (IUPAC Recommendations for Fundamental Stereochemistry (Section E), Pure Appl. Chem., 1976, 45:13-30)(incorporated by reference herein).

“Forms” means various isomers and mixtures thereof for a compound of Formula (I) or Formula (II). The term “isomer” refers to compounds that have the same composition and molecular weight but differ in physical and/or chemical properties. Such substances have the same number and kind of atoms but differ in structure. The structural difference may be in constitution (geometric isomers) or in an ability to rotate the plane of polarized light (stereoisomers). The term “stereoisomer” refers to isomers of identical constitution that differ in the arrangement of their atoms in space. Enantiomers and diastereomers are stereoisomers wherein an asymmetrically substituted carbon atom acts as a chiral center. The term “chiral” refers to a molecule that is not superposable on its mirror image, implying the absence of an axis and a plane or center of symmetry.

“Flat plasma curve” means a plasma concentration curve that reaches and maintains a substantially constant value after a defined period of time following administration of a dosage form according to the invention.

“Immediate-release dosage form” means a dosage form that releases greater than or equal to about 80% of the drug in less than or equal to about 1 hour following administration of the dosage form to a patient.

“Initiation of release” means the beginning of a release rate test, when the dosage form is placed in a liquid and the sequence of events begins that leads to release of the compounds of Formula (I) or of Formula (II).

“Medicament” means a product for use in preventing, treating or ameliorating substance related disorders such as substance dependence, substance abuse or substance induced disorders in a subject in need thereof.

“Oral sustained release dosing structure” means a structure suitable for oral administration to a patient comprising one or more compounds, wherein the structure operates to sustainably release the one or more compounds.

“Osmotic oral sustained release dosing structure” means an oral sustained release dosing structure wherein the structure operates via an osmotic mechanism to sustainably release one or more compounds.

“Patient” means an animal, preferably a mammal, more preferably a human, in need of therapeutic intervention.

“Pharmaceutically acceptable” means molecular entities and compositions that are of sufficient purity and quality for use in the formulation of a composition or medicament of the present invention. Since both human use (clinical and over-the-counter) and veterinary use are equally included within the scope of the present invention, a formulation would include a composition or medicament for either human or veterinary use.

“Pharmaceutically acceptable salt” means an acid or basic salt of the compounds of the invention that are of sufficient purity and quality for use in the formulation of a composition or medicament of the present invention and are tolerated and sufficiently non toxic to be used in a pharmaceutical preparation. Suitable pharmaceutically acceptable salts include acid addition salts which may, for example, be formed by reacting the drug compound with a suitable pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid.

Thus, representative pharmaceutically acceptable salts include, but are not limited to, the following: acetate, alpha-ketoglutarate, alpha-glycerophosphate, ascorbate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate; malate, maleate, malonate, mandelate, mesylate, methanesulfonate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate.

Pharmaceutically acceptable salts may be obtained using standard procedures well known in the art, for example by reacting a sufficiently basic compound such as an amine with a suitable acid affording a physiologically acceptable anion. Alkali metal, for example; sodium, potassium or lithium, or alkaline earth metals, for example calcium salts of carboxylic acids can also be made.

“Pharmacologically active metabolites” means pharmacologically active metabolites of drugs.

“Plasma drug concentration curve” or “drug plasma concentration curve”, or “plasma concentration curve” or “plasma profile” or “plasma concentration profile” refer to the curve obtained by plotting plasma drug concentration or drug plasma concentration, or plasma concentration versus time. Usually, the convention is that the zero point on the time scale (conventionally on the x axis) is the time of administration of the drug or dosage form comprising the drug to a patient.

“Prolonged period of time” means a continuous period of time of greater than about 2 hours, preferably, greater than about 4 hours, more preferably, greater than about 8 hours, more preferably greater than about 10 hours, more preferably still, greater than about 14 hours, most preferably, greater than about 14 hours and up to about 24 hours.

“Racemate” or “racemic mixture” means a compound of equimolar quantities of two enantiomeric species, wherein the compound is devoid of optical activity. The term “optical activity” refers to the degree to which a chiral molecule or nonracemic mixture of chiral molecules rotates the plane of polarized light.

“Rate of release” or “release rate” means to the quantity of compound released from a dosage form per unit time, e.g., milligrams of drug released per hour (mg/hr). Drug release rates for dosage forms may be measured as an in vitro rate of drug release, i.e., a quantity of drug released from the dosage form per unit time measured under appropriate conditions and in a suitable fluid.

The release rates referred to herein are determined by placing a dosage form to be tested in de-ionized water in metal coil or metal cage sample holders attached to a USP Type VII bath indexer in a constant temperature water bath at 37° C. Aliquots of the release rate solutions, collected at pre-set intervals, are then injected into a chromatographic system fitted with an ultraviolet or refractive index detector to quantify the amounts of drug released during the testing intervals.

As used herein a drug release rate obtained at a specified time refers to the in vitro release rate obtained at the specified time following implementation of the release rate test. The time at which a specified percentage of the drug within a dosage form has been released from said dosage form may be referred to as the “Tx” value, where “x” is the percent of drug that has been released. For example, a commonly used reference measurement for evaluating drug release from dosage forms is the time at which 70% of drug within the dosage form has been released. This measurement is referred to as the “T70” for the dosage form.

“Relative bioavailability” means AUC_{inf} for inventive dosage form/AUC_{inf} for immediate release dosage form;

wherein both dosage forms comprise the same or substantially the same amount of drug, expressed in units of mass.

“Steady state plasma concentration” means the condition in which the amount of drug present in the plasma of a patient does not vary significantly over a prolonged period of time. A pattern of drug accumulation following continuous administration of a constant dose and dosage form at constant dosing intervals eventually achieves a “steady-state” where the plasma concentration peaks and plasma concentration troughs are essentially identical from dosing interval to dosing interval. As used herein, the steady-state maximal (peak) plasma drug concentration obtained directly from the experimental data without interpolation is referenced as Cₘₐₓ₋ₛₛ and the steady-state minimal (trough) plasma drug concentration obtained directly from the experimental data without interpolation is referenced as Cₘᵢₙ₋ₛₛ. The times following drug administration at which the steady-state peak plasma and trough drug concentrations occur are referenced as the Tₘₐₓ₋ₛₛ and the Tₘᵢₙ₋ₛₛ, respectively. Typically these values are reported as a mean obtained over several patients or multiple administrations to the same patient, etc., once a steady state plasma concentration has been achieved.

“Sustained release” or “sustainably releasing” means continuous release or continuously releasing of a drug or a dose of a drug over a prolonged period of time.

“Mean, single dose, time to maximum plasma concentration Tmax” is the mean time elapsed from administration to a patient of a dosage form comprising a drug to the time at which the Cmax for that drug is obtained over several patients or multiple administrations to the same patient to the same patient with sufficient washout in between dosings to allow drug levels to subside to pre-dose levels, etc., following a single administration of the dosage form to each patient, and obtained directly from the experimental data without interpolation.

“Therapeutically effective amount” means that amount of drug that elicits the biological or medicinal response in a tissue system, animal or human that is being sought by a researcher, veterinarian, medical doctor or other clinician, which includes alleviation of the symptoms of the disease or disorder being treated.

“Zero order rate of release” or “zero order release rate” means a rate of release wherein the amount of drug released as a function of time is substantially constant. In other words, the dosage form exhibits zero order or substantially zero order release kinetics. More particularly, the rate of release of drug as a function of time shall vary by less than about 30%, preferably, less than about 20%, more preferably, less than about 10%, most preferably, less than about 5%, wherein the measurement is taken over the period of time wherein the cumulative release is between about 25% and about 75%, preferably, between about 25% and about 90%.

Plasma Profiles

The inventors have have noted certain side effects associated with administration of compounds of Formula (I) or Formula (II). These side effects include, but are not limited to headache, dizziness, euphoria, and nausea. The presence of the side effects may reduce patient compliance with administration of compounds of Formula (I) or Formula (II), because of the undesirable nature of these side effects.

The inventors have unexpectedly discovered that it is possible to adjust dosing of compounds of Formula (I) or Formula (II) such that side effects of these compounds can be reduced enough at clinically meaningful dosages so as to substantially reduce or substantially eliminate those side effects while maintaining enough drug in the body (as measured by plasma concentration) to presumably provide efficacy.

For instance, in Example 3 below, controlled release dosage forms according to the invention produced significantly less side effects as compared to classic immediate release dosing. The inventive pharmacokinetic profiles that accomplish this result, and dosage form release profiles that provide the inventive pharmacokinetic profiles, have not been demonstrated previously in the art.

Further, as noted above, the advantages of the present invention include reductions with regard to headache, dizziness, euphoria, nausea, and/or asthenia. An advantage of the present invention is that it provides for a new use for compounds of Formula (I) and of Formula (II), and of Formulas Ia, Ib, Iia, and IIb: preparation of a medicament for treatment of conditions responsive to treatment with such compounds while reducing, substantially reducing, eliminating or substantially eliminating the particular side effect(s)/condition(s) noted above.

In an embodiment, the invention provides for a reduction, substantial reduction, elimination or substantial elimination in the number of patients suffering from headache, dizziness, euphoria, nausea, and/or asthenia (including any one or any combination of these side effects) when receiving efficacious doses of compounds of Formula (I) or Formula (II) by about 15% or more compared to the patients taking an immediate release dosage form. In another embodiment, the invention provides for a reduction, substantial reduction, elimination or substantial elimination in the number of patients suffering from headache, dizziness, euphoria, nausea, and/or asthenia (including any one or any combination of these side effects) when receiving efficacious doses of compounds of Formula (I) or Formula (II) by about 30% or more compared to the patients taking an immediate release dosage form This new use is clinically meaningful for the reasons noted elsewhere herein.

Dosage Forms

In embodiments, the inventive sustained release dosage forms are formulated into dosage forms administrable to patients in need thereof. Sustained release dosage forms and methods of treatment using the sustained release dosage forms will now be described. It will be appreciated that the sustained release dosage forms described below are merely exemplary.

A variety of sustained release dosage forms are suitable for use in the present invention. In certain embodiments, the dosage form is orally administrable and is sized and shaped as a conventional tablet or capsule. Orally administrable dosage forms may be manufactured according to one of various different approaches. For example, the dosage form may be manufactured as a diffusion system, such as a reservoir device or matrix device, a dissolution system, such as encapsulated dissolution systems (including, for example, “tiny time pills”, and beads) and matrix dissolution systems, and combination diffusion/dissolution systems and ion-exchange resin systems, as described in Pharmaceutical Sciences, Remington, 18th Ed., pp. 1676-1686 (1990), Mack Publishing Co.; The Pharmaceutical and Clinical Pharmacokinetics, 3rd Ed., pp. 1-28 (1984), Lea and Febreger, Pa.

Osmotic dosage forms in general utilize osmotic pressure to generate a driving force for imbibing fluid into a compartment formed, at least in part, by a semipermeable membrane that permits free diffusion of fluid but not drug or osmotic agent(s), if present. A significant advantage to osmotic systems is that operation is pH-independent and thus continues at the osmotically determined rate throughout an extended time period even as the dosage form transits the gastrointestinal tract and encounters differing microenvironments having significantly different pH values. A review of such dosage forms is found in Santus and Baker, “Osmotic drug delivery: a review of the patent literature,” Journal of Controlled Release 35 (1995) 1-21, incorporated by reference herein. U.S. Pat. Nos. 3,845,770; 3,916,899; 3,995,631; 4,008,719; 4,111,202; 4,160,020; 4,327,725; 4,578,075; 4,681,583; 5,019,397; and 5,156,850 disclose osmotic devices that may be useful in the practice of the invention.

Osmotic dosage forms in which a drug composition may be delivered as a slurry, suspension or solution from a small exit orifice by the action of an expandable layer are disclosed in U.S. Pat. Nos. 5,633,011; 5,190,765; 5,252,338; 5,620,705; 4,931,285; 5,006,346; 5,024,842; and 5,160,743, which are incorporated herein by reference. Typical devices include an expandable push layer and a drug layer surrounded by a semipermeable membrane. In certain instances, the drug layer is provided with a subcoat to delay release of the drug composition to the environment of use or to form an annealed coating in conjunction with the semipermeable membrane.

A dosage form exhibiting substantially ascending release rate profile is Concerta® marketed by McNeil Consumer Healthcare and ALZA Pharmaceuticals. Physicians' Desk Reference, Thompson Healthcare, 56th Ed., pp.1998-2001 (2002). The Concerta® product, which contains methylphenidate as active agent, however, only delivers active agent at a substantially ascending rate of release for up to about 8 hours. Patent applications relating to Concerta® include published PCT Patent Application No. WO99/62496A1. This patent application discloses the substantially ascending release rate profile related to Concerta® for delivery over about 8 hours for once-a-day dosing.

Related ascending release rate profile patent applications include published PCT Patent Application No. WO98/14168; WO98/23263; WO98/06380A2,U.S.2001/0012847A1 and U.S.2002/0035357A1, which disclose an ascending release of active agent, including methylphenidate and pseudoephedrine, for up to about 8 hours. Still other applications relating to providing increasing rate of release delivery profile include WO01/52819A1, which discloses extended release of nifedipine and WO01/37813A2, which discloses at least a four layer preparation to provide controlled release.

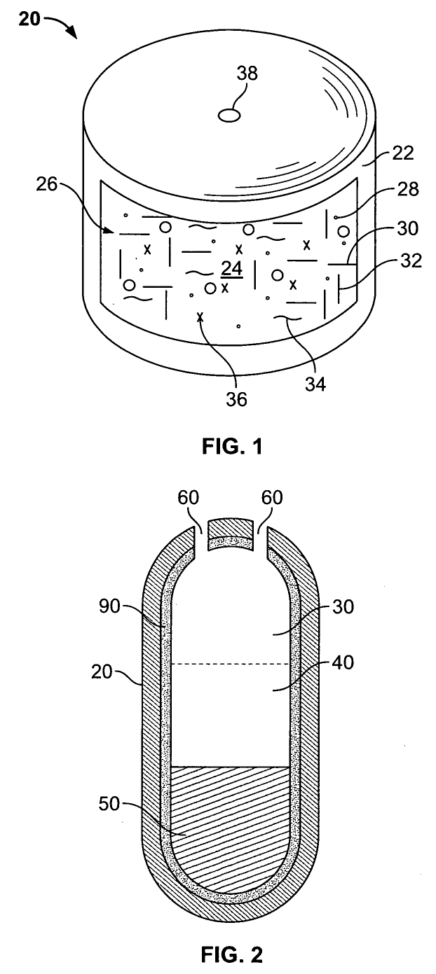

An exemplary dosage form, referred to in the art as an elementary osmotic pump dosage form, is shown in FIG. 1. Dosage form 20, shown in a cutaway view, is also referred to as an elementary osmotic pump, and is comprised of a semi-permeable wall 22 that surrounds and encloses an internal compartment 24. The internal compartment contains a single component layer referred to herein as drug layer 26, comprising a compound of Formula (I) or Formula (II) 28 in an admixture with selected excipients. The excipients are adapted to provide an osmotic activity gradient for attracting fluid from an external environment through wall 22 and for forming a deliverable complex formulation upon imbibition of fluid. The excipients may include a suitable suspending agent, also referred to herein as drug carrier 30, a binder 32, a lubricant 34, and an osmotically active agent referred to as an osmagent 36. Exemplary materials useful for these components can be found disclosed throughout the present application.

Semi-permeable wall 22 of the osmotic dosage form is permeable to the passage of an external fluid, such as water and biological fluids, but is substantially impermeable to the passage of components in the internal compartment. Materials useful for forming the wall are essentially nonerodible and are substantially insoluble in biological fluids during the life of the dosage form. Representative polymers for forming the semi-permeable wall include homopolymers and copolymers, such as, cellulose esters, cellulose ethers, and cellulose ester-ethers. Flux-regulating agents can be admixed with the wall-forming material to modulate the fluid permeability of the wall. For example, agents that produce a marked increase in permeability to fluid such as water are often essentially hydrophilic, while those that produce a marked permeability decrease to water are essentially hydrophobic. Exemplary flux regulating agents include polyhydric alcohols, polyalkylene glycols, polyalkylenediols, polyesters of alkylene glycols, and the like.

In operation, the osmotic gradient across wall 22 due to the presence of osmotically-active agents causes gastric fluid to be imbibed through the wall, swelling of the drug layer, and formation of a deliverable complex formulation (e.g., a solution, suspension, slurry or other flowable composition) within the internal compartment. The deliverable formulation comprising a compound of Formula (I) or Formula (II) is released through an exit 38 as fluid continues to enter the internal compartment. Even as drug formulation is released from the dosage form, fluid continues to be drawn into the internal compartment, thereby driving continued release. In this manner, the inventive substance is released in a sustained and continuous manner over an extended time period.

FIG. 2 illustrates certain inventive embodiments of sustained release dosage forms. Dosage forms of this type are described in detail in U.S. Pat. Nos. 4,612,008; 5,082,668; and 5,091,190; and are further described below

FIG. 2 shows an embodiment of one type of sustained release dosage form, namely the osmotic sustained release dosage form. First drug layer 30 comprises osmotically active components, and in certain embodiments a lower amount of drug than in second drug layer 40. The osmotically active component(s) in the first component drug layer comprises an osmagent such as salt and one or more osmopolymer(s) having relatively small molecular weights which exhibit swelling as fluid is imbibed such that release of these osmopolymers through exit 60 occurs similar to that of drug layer 40. Additional excipients such as binders, lubricants, antioxidants and colorants may also be included in first drug layer 30.

Second drug layer 40 comprises drug in an admixture with selected excipients adapted to provide an osmotic activity gradient for driving fluid from an external environment through membrane 20 and for forming a deliverable drug formulation upon imbibition of fluid. The excipients may include a suitable suspending agent, also referred to herein as a drug carrier, but no osmotically active agent, “osmagent,” such as salt, sodium chloride. It has been discovered that, in certain embodiments, the omission of salt from this second drug layer, which contains a higher proportion of the overall drug in the dosage form than first drug layer 30, in combination with the salt in the first drug layer, provides an improved ascending rate of release creating a longer duration of ascending rate.

The ratio of drug concentration between the first drug layer and the second drug layer alters the release rate profile. Release rate profile is calculated as the difference between the maximum release rate and the release rate achieved at the first time point after start-up (for example, at 6 hours), divided by the average release rate between the two data points. In an embodiment, drug layer 40 has a higher concentration of the drug than does drug layer 30. The ratio of the concentration of drug in the first drug layer 30 to the concentration of drug in the second drug layer 40 is maintained at less than 1 and preferably less than or equal to about 0.43 to provide the desired substantially ascending rate of release.

Drug layer 40 may also comprise other excipients such as lubricants, binders, etc.

Drug layer 40, as with drug layer 30, further comprises a hydrophilic polymer carrier. The hydrophilic polymer contributes to the controlled delivery of the active drug. Representative examples of these polymers are poly(alkylene oxide) of 100,000 to 750,000 number-average molecular weight, including poly(ethylene oxide), poly(methylene oxide), poly(butylene oxide) and poly(hexylene oxide); and a poly(carboxymethylcellulose) of 40,000 to 400,000 number-average molecular weight, represented by poly(alkali carboxymethylcellulose), poly(sodium carboxymethylcellulose), poly(potassium carboxymethylcellulose) and poly(lithium carboxymethylcellulose). Drug layer 40 can further comprise a hydroxypropylalkylcellulose of 9,200 to 125,000 number-average molecular weight for enhancing the delivery properties of the dosage form as represented by hydroxypropylethylcellulose, hydroxypropylmethylcellulose, hydroxypropylbutylcellulose and hydroxypropylpentylcellulose; and a poly(vinylpyrrolidone) of 7,000 to 75,000 number-average molecular weight for enhancing the flow properties of the dosage form. Preferred among these polymers are the poly(ethylene oxide) of 100,000-300,000 number average molecular weight. Carriers that erode in the gastric environment, i.e., bioerodible carriers, are especially preferred.

Other carriers that may be incorporated into drug layer 40, and/or drug layer 30, include carbohydrates that exhibit sufficient osmotic activity to be used alone or with other osmagents. Such carbohydrates comprise monosaccharides, disaccharides and polysaccharides. Representative examples include maltodextrins (i.e., glucose polymers produced by the hydrolysis of corn starch) and the sugars comprising lactose, glucose, raffinose, sucrose, mannitol, sorbitol, and the like. Preferred maltodextrins are those having a dextrose equivalence (DE) of 20 or less, preferably with a DE ranging from about 4 to about 20, and often 9-20. Maltodextrin having a DE of 9-12 has been found to be useful.

Drug layer 40 and drug layer 30 typically will be a substantially dry, <1% water by weight, composition formed by compression of the carrier, the drug, and other excipients as one layer.

Drug layer 40 may be formed from particles by comminution that produces the size of the drug and the size of the accompanying polymer used in the fabrication of the drug layer, typically as a core containing the compound, according to the mode and the manner of the invention. The means for producing particles include granulation, spray drying, sieving, lyophilization, crushing, grinding, jet milling, micronizing and chopping to produce the intended micron particle size. The process can be performed by size reduction equipment, such as a micropulverizer mill, a fluid energy grinding mill, a grinding mill, a roller mill, a hammer mill, an attrition mill, a chaser mill, a ball mill, a vibrating ball mill, an impact pulverizer mill, a centrifugal pulverizer, a coarse crusher and a fine crusher. The size of the particle can be ascertained by screening, including a grizzly screen, a flat screen, a vibrating screen, a revolving screen, a shaking screen, an oscillating screen and a reciprocating screen. The processes and equipment for preparing drug and carrier particles are disclosed in Pharmaceutical Sciences, Remington, 17th Ed., pp. 1585-1594 (1985); Chemical Engineers Handbook, Perry, 6th Ed., pp. 21-13 to 21-19 (1984); Journal of Pharmaceutical Sciences, Parrot, Vol. 61, No. 6, pp. 813-829 (1974); and Chemical Engineer, Hixon, pp. 94-103 (1990).

First drug layer 30 comprises drug in an admixture with selected excipients adapted to provide an osmotic activity gradient for driving fluid from an external environment through membrane 20 and for forming a deliverable drug formulation upon imbibition of fluid. The excipients may include a suitable suspending agent, also referred to herein as a drug carrier, and an osmotically active agent, i.e., an “osmagent,” such as salt. Other excipients such as lubricants, binders, etc. may also be included. The osmotically active component in the first drug layer typically comprises an osmagent and one or more osmopolymer(s) having relatively small molecular weights which exhibit swelling as fluid is imbibed such that release of these osmopolymers through exit 60 occurs similar to that of drug layer 40.

Drug layer 30 and drug layer 40 may optionally contain surfactants and disintegrants in both drug layers. Exemplary of the surfactants are those having an HLB value of about 10-25, such as polyethylene glycol 400 monostearate, polyoxyethylene4-sorbitan monolaurate, polyoxyethylene-20-sorbitan monooleate, polyoxyethylene-20-sorbitan monopalmitate, polyoxyethylene-20-monolaurate, polyoxyethylene40-stearate, sodium oleate and the like.

Disintegrants may be selected-from starches, clays, celluloses, algins and gums and crosslinked starches, celluloses and polymers. Representative disintegrants include corn starch, potato starch, croscarmelose, crospovidone, sodium starch glycolate, Veegum HV, methylcellulose, agar, bentonite, carboxymethylcellulose, alginic acid, guar gum and the like.

Membrane 20 is formed to be permeable to the passage of an external fluid, such as water and biological fluids, and is substantially impermeable to the passage of paliperidone, osmagent, osmopolymer and the like. As such, it is semipermeable. The selectively semipermeable compositions used for forming membrane 20 are essentially nonerodible and substantially insoluble in biological fluids during the life of the dosage form.

Representative polymers for forming membrane 20 comprise semipermeable homopolymers, semipermeable copolymers, and the like as disclosed generally herein. In one presently preferred embodiment, the compositions can comprise cellulose esters, cellulose ethers, and cellulose ester-ethers. The cellulosic polymers typically have a degree of substitution, “D.S.”, on their anhydroglucose unit from greater than 0 up to 3 inclusive. By degree of substitution is meant the average number of hydroxyl groups originally present on the anhydroglucose unit that are replaced by a substituting group, or converted into another group. The anhydroglucose unit can be partially or completely substituted with groups such as acyl, alkanoyl, alkenoyl, aroyl, alkyl, alkoxy, halogen, carboalkyl, alkylcarbamate, alkylcarbonate, alkylsulfonate, alkylsulfamate, semipermeable polymer forming groups, and the like. The semipermeable compositions typically include a member selected from the group consisting of cellulose acylate, cellulose diacylate, cellulose triacylate, cellulose triacetate, cellulose acetate, cellulose diacetate, cellulose triacetate, mono-, di- and tri-cellulose alkanylates, mono-, di-, and tri-alkenylates, mono-, di-, and tri-aroylates, and the like.

Exemplary polymers can include, for example, cellulose acetate have a D.S. of 1.8 to 2.3 and an acetyl content of 32 to 39.9%; cellulose diacetate having a D.S. of 1 to 2 and an acetyl content of 21 to 35%, cellulose triacetate having a D.S. of 2 to 3 and an acetyl content of 34 to 44.8%, and the like. More specific cellulosic polymers include cellulose propionate having a D.S. of 1.8 and a propionyl content of 38.5%; cellulose acetate propionate having an acetyl content of 1.5 to 7% and an acetyl content of 39 to 42%; cellulose acetate propionate having an acetyl content of 2.5 to 3%, an average propionyl content of 39.2 to 45%, and a hydroxyl content of 2.8 to 5.4%; cellulose acetate butyrate having a D.S. of 1.8, an acetyl content of 13 to 15%, and a butyryl content of 34 to 39%; cellulose acetate butyrate having an acetyl content of 2 to 29%, a butyryl content of 17 to 53%, and a hydroxyl content of 0.5 to 4.7%; cellulose triacylates having a D.S. of 2.6 to 3 such as cellulose trivalerate, cellulose trilamate, cellulose tripalmitate, cellulose trioctanoate, and cellulose tripropionate; cellulose diesters having a D.S. of 2.2 to 2.6 such as cellulose disuccinate, cellulose dipalmitate, cellulose dioctanoate, cellulose dicarpylate, and the like; mixed cellulose esters such as cellulose acetate valerate, cellulose acetate succinate, cellulose propionate succinate, cellulose acetate octanoate, cellulose valerate palmitate, cellulose acetate heptonate, and the like. Semipermeable polymers are known in U.S. Pat. No. 4,077,407 and they can be synthesized by procedures described in Encyclopedia of Polymer Science and Technology, Vol. 3, pages 325 to 354, 1964, published by Interscience Publishers, Inc., New York.

Additional semipermeable polymers for forming the semipermeable wall can comprise, for example, cellulose acetaldehyde dimethyl acetate; cellulose acetate ethylcarbamate; cellulose acetate methylcarbamate; cellulose dimethylaminoacetate; semipermeable polyamide; semipermeable polyurethanes; semipermeable sulfonated polystyrenes; cross-linked selectively semipermeable polymers formed by the coprecipitation of a polyanion and a polycation as disclosed in U.S. Pat. Nos. 3,173,876; 3,276,586; 3,541,005; 3,541,006; and 3,546,142; semipermeable polymers as disclosed in U.S. Pat. No. 3,133,132; semipermeable polystyrene derivatives; semipermeable poly (sodium styrenesulfonate); semipermeable poly (vinylbenzyltremethylammonium chloride); semipermeable polymers, exhibiting a fluid permeability of 10-5 to 10-2 (cc. mil/cm hr.atm) expressed as per atmosphere of hydrostatic or osmotic pressure differences across a semipermeable wall. The polymers are known to the art in U.S. Pat. Nos. 3,845,770; 3,916,899; and 4,160,020; and in Handbook of Common Polymers, by Scott, J. R., and Roff, W. J., 1971, published by CRC Press, Cleveland. Ohio.

Wall 20 may also comprise a flux-regulating agent. The flux regulating agent is a compound added to assist in regulating the fluid permeability or flux through the wall 20. The flux regulating agent can be a flux enhancing agent or a decreasing agent. The agent can be preselected to increase or decrease the liquid flux. Agents that produce a marked increase in permeability to fluids such as water are often essentially hydrophilic, while those that produce a marked decrease to fluids such as water are essentially hydrophobic. The amount of regulator in wall 20 when incorporated therein generally is from about 0.01% to 20% by weight or more. The flux regulator agents in one embodiment that increase flux include, for example, polyhydric alcohols, polyalkylene glycols, polyalkylenediols, polyesters of alkylene glycols, and the like. Typical flux enhancers include polyethylene glycol 300, 400, 600, 1500, 4000, 6000, poly(ethylene glycol-co-propylene glycol), and the like; low molecular weight gylcols such as polypropylene glycol, polybutylene glycol and polyamylene glycol: the polyalkylenediols such as poly(1,3-propanediol), poly(1,4-butanediol), poly(1,6-hexanediol), and the like; aliphatic diols such as 1,3-butylene glycol, 1,4-pentamethylene glycol, 1,4-hexamethylene glycol, and the like; alkylene triols such as glycerine, 1,2,3-butanetriol, 1,2,4-hexanetriol, 1,3,6-hexanetriol and the like; esters such as ethylene glycol dipropionate, ethylene glycol butyrate, butylene glucol dipropionate, glycerol acetate esters, and the like, including those disclosed elsewhere herein. Representative flux decreasing agents include, for example, phthalates substituted with an alkyl or alkoxy or with both an alkyl and alkoxy group such as diethyl phthalate, dimethoxyethyl phthalate, dimethyl phthalate, and [di(2-ethylhexyl)phthalate], aryl phthalates such as triphenyl phthalate, and butyl benzyl phthalate; insoluble salts such as calcium sulphate, barium sulphate, calcium phosphate, and the like; insoluble oxides such as titanium oxide; polymers in powder, granule and like form such as polystyrene, polymethylmethacrylate, polycarbonate, and polysulfone; esters such as citric acid esters esterfied with long chain alkyl groups; inert and substantially water impermeable fillers; resins compatible with cellulose based wall forming materials, and the like.

Other materials that can be used to form wall 20 for imparting flexibility and elongation properties to the wall, for making the wall less-to-nonbrittle and to render tear strength, include, for example, phthalate plasticizers such as dibenzyl phthalate, dihexyl phthalate, butyl octyl phthalate, straight chain phthalates of six to eleven carbons, di-isononyl phthalte, di-isodecyl phthalate, and the like. The plasticizers include nonphthalates such as triacetin, dioctyl azelate, epoxidized tallate, tri-isoctyl trimellitate, tri-isononyl trimellitate, sucrose acetate isobutyrate, epoxidized soybean oil, and the like. The amount of plasticizer in a wall when incorporated therein is about 0.01% to 20% weight, or higher.

Push layer 50 comprises an expandable layer in contacting layered arrangement with the second component drug layer 40 as illustrated in FIG. 2. Push layer 50 comprises a polymer that imbibes an aqueous or biological fluid and swells to push the drug composition through the exit of the device.

The expandable layer comprises in one embodiment a hydroactivated composition that swells in the presence of water, such as that present in gastric fluids. Conveniently, it can comprise an osmotic composition comprising an osmotic solute that exhibits an osmotic pressure gradient across the semipermeable layer against an external fluid present in the environment of use. In another embodiment, the hydro-activated layer comprises a hydrogel that imbibes and/or absorbs fluid into the layer through the outer semipermeable wall. The semipermeable wall is non-toxic. It maintains its physical and chemical integrity during operation and it is essentially free of interaction with the expandable layer.

The expandable layer in one preferred embodiment comprises a hydroactive layer comprising a hydrophilic polymer, also known as osmopolymers. The osmopolymers exhibit fluid imbibition properties. The osmopolymers are swellable, hydrophilic polymers, which osmopolymers interact with water and biological aqueous fluids and swell or expand to an equilibrium state. The osmopolymers exhibit the ability to swell in water and biological fluids and retain a significant portion of the imbibed fluid within the polymer structure. The osmopolymers swell or expand to a very high degree, usually exhibiting a 2 to 50 fold volume increase. The osmopolymers can be non-cross-linked or cross-linked. The swellable, hydrophilic polymers are in one embodiment lightly cross-linked, such cross-links being formed by covalent or ionic bonds or residue crystalline regions after swelling. The osmopolymers can be of plant, animal or synthetic origin.

The osmopolymers are hydrophilic polymers, and are disclosed generally throughout the present disclosure. Hydrophilic polymers suitable for the present purpose include poly (hydroxy-alkyl methacrylate) having a molecular weight of from 30,000 to 5,000,000; poly (vinylpyrrolidone) having a molecular weight of from 10,000 to 360,000; anionic and cationic hydrogels; polyelectrolytes complexes; poly (vinyl alcohol) having a low acetate residual, cross-linked with glyoxal, formaldehyde, or glutaraldehyde and having a degree of polymerization of from 200 to 30,000; a mixture of methyl cellulose, cross-linked agar and carboxymethyl cellulose; a mixture of hydroxypropyl methylcellulose and sodium carboxymethylcellulose; a mixture of hydroxypropyl ethylcellulose and sodium carboxymethyl cellulose, a mixture of sodium carboxymethylcellulose and methylcellulose, sodium carboxymethylcellulose; potassium carboxymethylcellulose; a water insoluble, water swellable copolymer formed from a dispersion of finely divided copolymer of maleic anhydride with styrene, ethylene, propylene, butylene or isobutylene crosslinked with from 0.001 to about 0.5 moles of saturated cross-linking agent per mole of maleic anhydride per copolymer; water swellable polymers of N-vinyl lactams; polyoxyethylene-polyoxypropylene gel; carob gum; polyacrylic gel; polyester gel; polyuria gel; polyether gel, polyamide gel; polycellulosic gel; polygum gel; initially dry hydrogels that imbibe and absorb water which penetrates the glassy hydrogel and lowers its glass temperature; and the like.

Representative of other osmopolymers are polymers that form hydrogels such as Carbopol™. acidic carboxypolymer, a polymer of acrylic acid cross-linked with a polyallyl sucrose, also known as carboxypolymethylene, and carboxyvinyl polymer having a molecular weight of 250,000 to 4,000,000; Cyanamer™ polyacrylamides; cross-linked water swellable indenemaleic anhydride polymers; Good-rite™ polyacrylic acid having a molecular weight of 80,000 to 200,000; Polyox™ polyethylene oxide polymer having a molecular weight of 100,000 to 5,000,000 and higher; starch graft copolymers; Aqua-Keeps™ acrylate polymer polysaccharides composed of condensed glucose units such as diester cross-linked polygluran; and the like. Representative polymers that form hydrogels are known to the prior art in U.S. Pat. No. 3,865,108; U.S. Pat. No. 4,002,173; U.S. Pat. No. 4,207,893; and in Handbook of Common Polymers, by Scott and Roff, published by the Chemical Rubber Co., Cleveland, Ohio. The amount of osmopolymer comprising a hydro-activated layer can be from about 5% to 100%.

The expandable layer in another manufacture can comprise an osmotically effective compound that comprises inorganic and organic compounds that exhibit an osmotic pressure gradient across a semipermeable wall against an external fluid. The osmotically effective compounds, as with the osmopolymers, imbibe fluid into the osmotic system, thereby making available fluid to push against the inner wall, i.e., in some embodiments, the barrier layer and/or the wall of the soft or hard capsule for pushing drug from the dosage form. The osmotically effective compounds are known also as osmotically effective solutes, and also as osmagents. Osmotically effective solutes that can be used comprise magnesium sulfate, magnesium chloride, potassium sulfate, sodium sulfate, lithium sulfate, potassium acid phosphate, mannitol, urea, inositol, magnesium succinate, tartaric acid, carbohydrates such as raffinose, sucrose, glucose, lactose, sorbitol, and mixtures therefor. The amount of osmagent in can be from about 5% to 100% of the weight of the layer. The expandable layer optionally comprises an osmopolymer and an osmagent with the total amount of osmopolymer and osmagent equal to 100%. Osmotically effective solutes are known to the prior art as described in U.S. Pat. No. 4,783,337.

Protective subcoat, inner wall 90, is permeable to the passage of gastric fluid entering the compartment defined by wall 20 and provides a protective function that reduces the degradation of drug under stress conditions.

Inner wall 90 further provides a lubricating function that facilitates the movement of first drug layer 30, second drug layer 40 and push layer 50 toward exit 60. Inner wall 90 may be formed from hydrophilic materials and excipients. Outer wall 20 is semipermeable, allowing gastric fluid to enter the compartment, but preventing the passage of the materials comprising the core in the compartment. The deliverable drug formulation is released from exit 60 upon osmotic operation of the osmotic oral dosage form.

Inner wall 90 also reduces friction between the external surface of drug layer 30 and drug layer 40, and the inner surface of wall 20. Inner wall 90 promotes release of the drug composition from the compartment and reduces the amount of residual drug composition remaining in the compartment at the end of the delivery period, particularly when the slurry, suspension or solution of the drug composition that is being dispensed is highly viscous during the period of time in which it is being dispensed. In dosage forms with hydrophobic agents and no inner wall, it has been observed that significant residual amounts of drug may remain in the device after the period of delivery has been completed. In some instances, amounts of 20% or greater may remain in the dosage form at the end of a twenty-four hour period when tested in a release rate assay.

Inner wall 90 is formed as an inner coat of a flow-promoting agent, i.e., an agent that lowers the frictional force between the outer wall 20 and the external surface of drug layer 40. Inner wall 90 appears to reduce the frictional forces between outer wall 20 and the outer surface of drug layer 30 and drug layer 40, thus allowing for more complete delivery of drug from the device. Particularly in the case of active compounds having a high cost, such an improvement presents substantial economic advantages since it is not necessary to load the drug layer with an excess of drug to insure that the minimum amount of drug required will be delivered. Inner wall 90 may be formed as a coating applied over the compressed core.

Inner wall 90 is further characterized by an optional protective agent, i.e., an agent that reduces the degradation of drug in drug layer 30 and drug layer 40. Particularly in the case of active compounds having a high cost, such an improvement presents substantial economic advantages. Inner wall 90 may be formed as a coating applied over the compressed core.

Inner wall 90 typically may be 0.01 to 5 mm thick, more typically 0.5 to 5 mm thick, and it comprises a member selected from hydrogels, gelatin, low molecular weight polyethylene oxides, e.g., less than 100,000 MW, hydroxyalkylcelluloses, e.g., hydroxyethylcellulose, hydroxypropylcellulose, hydroxyisopropylcelluose, hydroxybutylcellulose and hydroxyphenylcellulose, and hydroxyalkyl alkylcelluloses, e.g., hydroxypropyl methylcellulose, and mixtures thereof. The hydroxyalkylcelluloses comprise polymers having a 9,500 to 1,250,000 number-average molecular weight. For example, hydroxypropyl celluloses having number average molecular weights of 80,000 to 850,000 are useful. The inner wall may be prepared from conventional solutions or suspensions of the aforementioned materials in aqueous solvents or inert organic solvents.

Prefered materials for the inner wall include hydroxypropyl cellulose, hydroxyethyl cellulose, hydroxypropyl methyl cellulose, povidone [poly(vinylpyrrolidone)], polyethylene glycol, and mixtures thereof.

Most prefered are mixtures of hydroxypropyl cellulose and povidone, prepared in organic solvents, particularly organic polar solvents such as lower alkanols having 1-8 carbon atoms, preferably ethanol, mixtures of hydroxyethyl cellolose and hydroxypropyl methyl cellulose prepared in aqueous solution, and mixtures of hydroxyethyl cellulose and polyethylene glycol prepared in aqueous solution. Most preferably, the inner wall comprises a mixture of hydroxypropyl cellulose and providone prepared in ethanol.

It is preferred that inner wall 90 comprises between about 50% and about 90% hydroxypropylcellulose identified as EF having an average molecular weight of about 80,000 and between about 10% and about 50% polyvinylpyrrolidone identified as K29-32.

Conveniently, the weight of the inner wall applied to the compressed core may be correlated with the thickness of the inner wall and residual drug remaining in a dosage form in a release rate assay such as described herein. As such, during manufacturing operations, the thickness of the inner wall may be controlled by controlling the weight of the inner wall taken up in the coating operation.

When inner wall 90 is formed as a subcoat, i.e., by coating onto the tabletted composite including one or all of the first drug layer, second drug layer and push layer, the inner wall can fill in surface irregularities formed on the core by the tabletting process. The resulting smooth external surface facilitates slippage between the coated composite core and the semipermeable wall during dispensing of the drug, resulting in a lower amount of residual drug composition remaining in the device at the end of the dosing period. When inner wall 90 is fabricated of a gel-forming material, contact with water in the environment of use facilitates formation of the gel or gel-like inner coat having a viscosity that may promote and enhance slippage between outer wall 20 and drug layer 30 and drug layer 40.

Pan coating may be conveniently used to provide the completed dosage form, except for the exit orifice. In the pan coating system, the wall-forming composition for the inner wall or the outer wall, as the case may be, is deposited by successive spraying of the appropriate wall composition onto the compressed trilayered or multilayered core comprising the drug layers, optional barrier layer and push layer, accompanied by tumbling in a rotating pan. A pan coater is used because of its availability at commercial scale. Other techniques can be used for coating the compressed core. Once coated, the wall is dried in a forced-air oven or in a temperature and humidity controlled oven to free the dosage form of solvent(s) used in the manufacturing. Drying conditions will be conventionally chosen on the basis of available equipment, ambient conditions, solvents, coatings, coating thickness, and the like.

Other coating techniques can also be employed. For example, the wall or walls of the dosage form may be formed in one technique using the air-suspension procedure. This procedure consists of suspending and tumbling the compressed core in a current of air and the semipermeable wall forming composition, until the wall is applied to the core. The air-suspension procedure is well suited for independently forming the wall of the dosage form. The air-suspension procedure is described in U.S. Pat. No. 2,799,241; in J. Am. Pharm. Assoc., Vol. 48, pp. 451459 (1959); and, ibid., Vol. 49, pp. 82-84 (1960). The dosage form also can be coated with a Wurster® air-suspension coater using, for example, methylene dichloride methanol as a cosolvent for the wall forming material. An Aeromatic® air-suspension coater can be used employing a cosolvent.

In an embodiment, the sustained release dosage form of the invention is provided with at least one exit 60 as shown in FIG. 2. Exit 60 cooperates with the compressed core for the uniform release of drug from the dosage form. The exit can be provided during the manufacture of the dosage form or during drug delivery by the dosage form in a fluid environment of use.

One or more exit orifices are drilled in the drug layer end of the dosage form, and optional water soluble overcoats, which may be colored (e.g., Opadry colored coatings) or clear (e.g., Opadry Clear), may be coated on the dosage form to provide the finished dosage form.

Exit 60 may include an orifice that is formed or formable from a substance or polymer that erodes, dissolves or is leached from the outer wall to thereby form an exit orifice. The substance or polymer may include, for example, an erodible poly(glycolic) acid or poly(lactic) acid in the semipermeable wall; a gelatinous filament; a water-removable poly(vinyl alcohol); a leachable compound, such as a fluid removable pore-former selected from the group consisting of inorganic and organic salt, oxide and carbohydrate.

An exit, or a plurality of exits, can be formed by leaching a member selected from the group consisting of sorbitol, lactose, fructose, glucose, mannose, galactose, talose, sodium chloride, potassium chloride, sodium citrate and mannitol to provide a uniform-release dimensioned pore-exit orifice.

The exit can have any shape, such as round, triangular, square, elliptical and the like for the uniform metered dose release of a drug from the dosage form.

The sustained release dosage form can be constructed with one or more exits in spaced-apart relation or one or more surfaces of the sustained release dosage form.

Drilling, including mechanical and laser drilling, through the semipermeable wall can be used to form the exit orifice. Such exits and equipment for forming such exits are disclosed in U.S. Pat. No. 3,916,899, by Theeuwes and Higuchi and in U.S. Pat. No. 4,088,864, by Theeuwes, et al. It is presently preferred to utilize two exits of equal diameter. In a preferred embodiment, exit 60 penetrates through subcoat 90, if present, to drug layer 30.

Dosage forms in accordance with the embodiments depicted in FIG. 1 are manufactured by standard techniques. For example, the dosage form may be manufactured by the wet granulation technique. In the wet granulation technique, the drug and carrier are blended using an organic solvent, such as denatured anhydrous ethanol, as the granulation fluid. The remaining ingredients can be dissolved in a portion of the granulation fluid, such as the solvent described above, and this latter prepared wet blend is slowly added to the drug blend with continual mixing in the blender. The granulating fluid is added until a wet blend is produced, which wet mass blend is then forced through a predetermined screen onto oven trays. The blend is dried for 18 to 24 hours at 24° C. to 35° C. in a forced-air oven. The dried granules are then sized. Next, magnesium stearate, or another suitable lubricant, is added to the drug granulation, and the granulation is put into milling jars and mixed on a jar mill for 10 minutes. The composition is pressed into a layer, for example, in a Manesty® press or a Korsch LCT press. For a trilayered core, granules or powders of the drug layer compositions and push layer composition are sequentially placed in an appropriately-sized die with intermediate compression steps being applied to each of the first two layers, followed by a final compression step after the last layer is added to the die to form the trilayered core. The intermediate compression typically takes place under a force of about 50-100 newtons. Final stage compression typically takes place at a force of 3500 newtons or greater, often 3500-5000 newtons. The compressed cores are fed to a dry coater press, e.g., Kilian® Dry Coater press, and subsequently coated with the wall materials as described above.

In another embodiment, the drug and other ingredients comprising the drug layer are blended and pressed into a solid layer. The layer possesses dimensions that correspond to the internal dimensions of the area the layer is to occupy in the dosage form, and it also possesses dimensions corresponding to the push layer, if included, for forming a contacting arrangement therewith. The drug and other ingredients can also be blended with a solvent and mixed into a solid or semisolid form by conventional methods, such as ballmilling, calendering, stirring or rollmilling, and then pressed into a preselected shape. Next, if included, a layer of osmopolymer composition is placed in contact with the layer of drug in a like manner. The layering of the drug formulation and the osmopolymer layer can be fabricated by conventional two-layer press techniques. An analogous procedure may be followed for the preparation of the trilayered core. The compressed cores then may be coated with the inner wall material and the semipermeable wall material as described above.

Another manufacturing process that can be used comprises blending the powdered ingredients for each layer in a fluid bed granulator. After the powdered ingredients are dry blended in the granulator, a granulating fluid, for example, poly(vinylpyrrolidone) in water, is sprayed onto the powders. The coated powders are then dried in the granulator. This process granulates all the ingredients present therein while adding the granulating fluid. After the granules are dried, a lubricant, such as stearic acid or magnesium stearate, is mixed into the granulation using a blender e.g., V-blender or tote blender. The granules are then pressed in the manner described above.

Exemplary solvents suitable for manufacturing the dosage form components comprise aqueous or inert organic solvents that do not adversely harm the materials used in the system. The solvents broadly include members selected from the group consisting of aqueous solvents, alcohols, ketones, esters, ethers, aliphatic hydrocarbons, halogenated solvents, cycloaliphatics, aromatics, heterocyclic solvents and mixtures thereof. Typical solvents include acetone, diacetone alcohol, methanol, ethanol, isopropyl alcohol, butyl alcohol, methyl acetate, ethyl acetate, isopropyl acetate, n-butyl acetate, methyl isobutyl ketone, methyl propyl ketone, n-hexane, n-heptane, ethylene glycol monoethyl ether, ethylene glycol monoethyl acetate, methylene dichloride, ethylene dichloride, propylene dichloride, carbon tetrachloride nitroethane, nitropropane tetrachloroethane, ethyl ether, isopropyl ether, cyclohexane, cyclooctane, benzene, toluene, naphtha, 1,4-dioxane, tetrahydrofuran, diglyme, water, aqueous solvents containing inorganic salts such as sodium chloride, calcium chloride, and the like, and mixtures thereof such as acetone and water, acetone and methanol, acetone and ethyl alcohol, methylene dichloride and methanol, and ethylene dichloride and methanol.

One important consideration in the practice of this invention is the physical state of the compound to be delivered by the dosage form. In certain embodiments, the compounds may be in a paste or liquid state. In such cases solid dosage forms may not be suitable for use in the practice of this invention. Instead, dosage forms capable of delivering substances in a paste or liquid state should be used.

The present invention provides a liquid formulation of compounds of Formula (I) or Formula (II) for use with oral delivery devices. Oral osmotic devices for delivering liquid formulations and methods of using them are known in the art, for example, as described and claimed in the following U.S. Pat. Nos. owned by ALZA corporation: 6,419,952; 6,174,547; 6,551,613; 5,324,280; 4,111,201; and 6,174,547. Methods of using oral osmotic devices for delivering therapeutic agents at an ascending rate of release can be found in International Application Numbers WO 98/06380, WO 98/23263, and WO 99/62496.

Exemplary liquid carriers for the present invention include lipophilic solvents (e.g., oils and lipids), surfactants, and hydrophilic solvents. Exemplary lipophilic solvents, for example, include, but are not limited to, Capmul PG-8, Caprol MPGO, Capryol 90, Plurol Oleique CC 497, Capmul MCM, Labrafac PG, N-Decyl Alcohol, Caprol 10O10O, Oleic Acid, Vitamin E, Maisine 35-1, Gelucire 33/01, Gelucire 44/14, Lauryl Alcohol, Captex 355EP, Captex 500, Capylic/Caplic Triglyceride, Peceol, Caprol ET, Labrafil M2125 CS, Labrafac CC, Labrafil M 1944 CS, Captex 8277, Myvacet 9-45, Isopropyl Nyristate, Caprol PGE 860, Olive Oil, Plurol Oleique, Peanut Oil, Captex 300 Low C6, and Capric Acid.

Exemplary surfactants for example, include, but are not limited to, Vitamin E TPGS, Cremophor (grades EL, EL-P, and RH40), Labrasol, Tween (grades 20, 60, 80), Pluronic (grades L-31, L-35, L-42, L-64, and L-121), Acconon S-35, Solutol HS-15, and Span (grades 20, and 80). Exemplary hydrophilic solvents for example, include, but are not limited to, Isosorbide Dimethyl Ether, Polyethylene Glycol (PEG grades 300, 400, 600, 3000, 4000, 6000, and 8000) and Propylene Glycol (PG).

The skilled practitioner will understand that any formulation comprising a sufficient dosage of a compound of Formula (I) or Formula (II) solubilized in a liquid carrier suitable for administration to a subject and for use in an osmotic device can be used in the present invention. In one exemplary embodiment of the present invention, the liquid carrier is PG, Solutol, Cremophor EL, or a combination thereof.

The liquid formulation according to the present invention can also comprise, for example, additional excipients such as an antioxidant, permeation enhancer and the like. Antioxidants can be provided to slow or effectively stop the rate of any autoxidizable material present in the capsule. Representative antioxidants can comprise a member selected from the group of ascorbic acid; alpha tocopherol; ascorbyl palmitate; ascorbates; isoascorbates; butylated hydroxyanisole; butylated hydroxytoluene; nordihydroguiaretic acid; esters of garlic acid comprising at least 3 carbon atoms comprising a member selected from the group consisting of propyl gallate, octyl gallate, decyl gallate, decyl gallate; 6-ethoxy-2,2,4-trimethyl-1,2-dihydro-guinoline; N-acetyl-2,6-di-t-butyl-p-aminophenol; butyl tyrosine; 3-tertiarybutyl-4-hydroxyanisole; 2-tertiary-butyl-4-hydroxyanisole; 4-chloro-2,6-ditertiary butyl phenol; 2,6-ditertiary butyl p-methoxy phenol; 2,6-ditertiary butyl-p-cresol: polymeric antioxidants; trihydroxybutyro-phenone physiologically acceptable salts of ascorbic acid, erythorbic acid, and ascorbyl acetate; calcium ascorbate; sodium ascorbate; sodium bisulfite; and the like. The amount of antioxidant used for the present purposes, for example, can be about 0.001% to 25% of the total weight of the composition present in the lumen. Antioxidants are known to the prior art in U.S. Pat. Nos. 2,707,154; 3,573,936; 3,637,772; 4,038,434; 4,186,465 and 4,559,237, each of which is hereby incorporated by reference in its entirety for all purposes.

The inventive liquid formulation can comprise permeation enhancers that facilitate absorption of the drug in the environment of use. Such enhancers can, for example, open the so-called “tight junctions” in the gastrointestinal tract or modify the effect of cellular components, such a p-glycoprotein and the like. Suitable enhancers can include alkali metal salts of salicyclic acid, such as sodium salicylate, caprylic or capric acid, such as sodium caprylate or sodium caprate, and the like. Enhancers can include, for example, the bile salts, such as sodium deoxycholate. Various p-glycoprotein modulators are described in U.S. Pat. Nos. 5,112,817 and 5,643,909. Various other absorption enhancing compounds and materials are described in U.S. Pat. No. 5,824,638. Enhancers can be used either alone or as mixtures in combination with other enhancers.

In certain embodiments, compounds of Formula (I) or Formula (II) may be administered as a self-emulsifying formulation. Like the other liquid carriers, the surfactant functions to prevent aggregation, reduce interfacial tension between constituents, enhance the free-flow of constituents, and lessen the incidence of constituent retention in the dosage form. The emulsion formulation of this invention comprises a surfactant that imparts emulsification. Exemplary surfactants can also include, for example, in addition to the surfactants listed above, a member selected from the group consisting of polyoxyethylenated castor oil comprising ethylene oxide in the concentration of 9 to 15 moles, polyoxyethylenated sorbitan monopalmitate, mono and tristearate comprising 20 moles of ethylene oxide, polyoxyethylenated sorbitan monostearate comprising 4 moles of ethylene oxide, polyoxyethylenated sorbitan trioleate comprising 20 moles of ethylene oxide, polyoxyethylene lauryl ether, polyoxyethylenated stearic acid comprising 40 to 50 moles of ethylene oxide, polyoxyethylenated stearyl alcohol comprising 2 moles of ethylene oxide, and polyoxyethylenated oleyl alcohol comprising 2 moles of ethylene oxide. The surfactants may be available from Atlas Chemical Industries.

The drug emulsified formulations of the present invention can initially comprise an oil and a non-ionic surfactant. The oil phase of the emulsion comprises any pharmaceutically acceptable oil which is not immiscible with water. The oil can be an edible liquid such as a non-polar ester of an unsaturated fatty acid, derivatives of such esters, or mixtures of such esters. The oil can be vegetable, mineral, animal or marine in origin. Examples of non-toxic oils can also include, for example, in addition to the surfactants listed above, a member selected from the group consisting of peanut oil, cottonseed oil, sesame oil, corn oil, almond oil, mineral oil, castor oil, coconut oil, palm oil, cocoa butter, safflower, a mixture of mono- and diglycerides of 16 to 18 carbon atoms, unsaturated fatty acids, fractionated triglycerides derived from coconut oil, fractionated liquid triglycerides derived from short chain 10 to 15 carbon atoms fatty acids, acetylated monoglycerides, acetylated diglycerides, acetylated triglycerides, olein known also as glyceral trioleate, palmitin known as glyceryl tripalmitate, stearin known also as glyceryl tristearate, lauric acid hexylester, oleic acid oleylester, glycolyzed ethoxylated glycerides of natural oils, branched fatty acids with 13 molecules of ethyleneoxide, and oleic acid decylester. The concentration of oil, or oil derivative in the emulsion formulation can be from about 1 wt % to about 40 wt %, with the wt % of all constituents in the emulsion preparation equal to 100 wt %. The oils are disclosed in Pharmaceutical Sciences by Remington, 17th Ed., pp. 403-405, (1985) published by Mark Publishing Co., in Encyclopedia of Chemistry, by Van Nostrand Reinhold, 4th Ed., pp. 644-645, (1984) published by Van Nostrand Reinhold Co.; and in U.S. Pat. No. 4,259,323.

The amount of compounds of Formula (I) or Formula (II) incorporated in the dosage forms of the present invention is generally from about 10% to about 90% by weight of the composition depending upon the therapeutic indication and the desired administration period, e.g., every 12 hours, every 24 hours, and the like. Depending on the dose of a compound of Formula (I) or Formula (II) desired to be administered, one or more of the dosage forms can be administered.

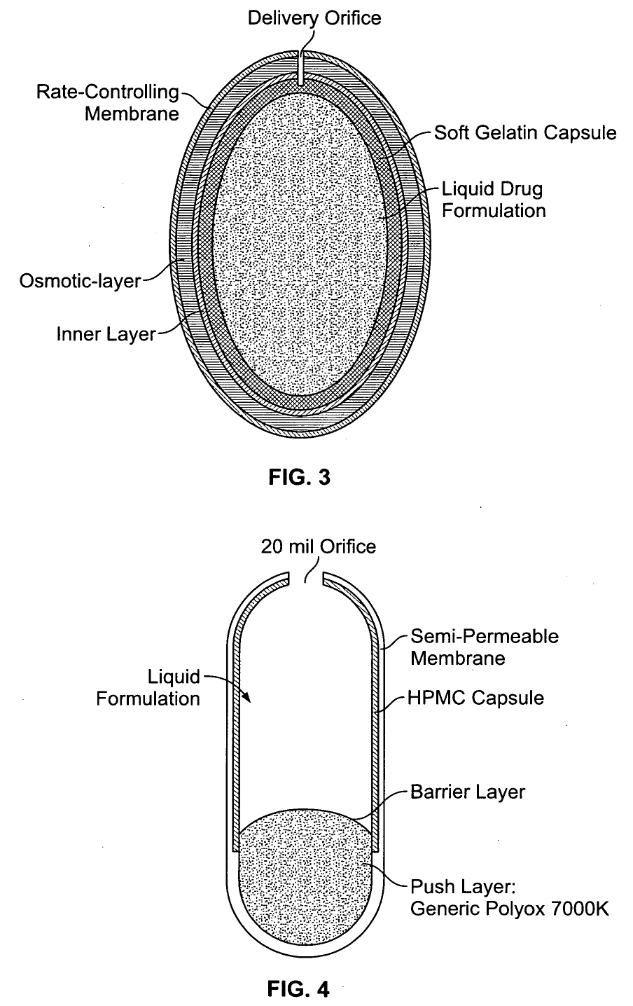

The osmotic dosage forms of the present invention can possess two distinct forms, a soft capsule form (shown in FIG. 4) and a hard capsule form (shown in FIG. 3). The soft capsule, as used by the present invention, preferably in its final form comprises one piece. The one-piece capsule is of a sealed construction encapsulating the drug formulation therein. The capsule can be made by various processes including the plate process, the rotary die process, the reciprocating die process, and the continuous process. An example of the plate process is as follows. The plate process uses a set of molds. A warm sheet of a prepared capsule lamina-forming material is laid over the lower mold and the formulation poured on it. A second sheet of the lamina-forming material is placed over the formulation followed by the top mold. The mold set is placed under a press and a pressure applied, with or without heat, to form a unit capsule. The capsules are washed with a solvent for removing excess formulation from the exterior of the capsule, and the air-dried capsule is encapsulated with a semipermeable wall. The rotary die process uses two continuous films of capsule lamina-forming material that are brought into convergence between a pair of revolving dies and an injector wedge. The process fills and seals the capsule in dual and coincident operations. In this process, the sheets of capsule lamina-forming material are fed over guide rolls, and then down between the wedge injector and the die rolls. The drug formulation to be encapsulated flows by gravity into a positive displacement pump. The pump meters the drug formulation through the wedge injector and into the sheets between the die rolls. The bottom of the wedge contains small orifices lined up with the die pockets of the die rolls. The capsule is about half-sealed when the pressure of pumped drug formulation forces the sheets into the die pockets, wherein the capsules are simultaneously filled, shaped, hermetically sealed and cut from the sheets of lamina-forming materials. The sealing of the capsule is achieved by mechanical pressure on the die rolls and by heating of the sheets of lamina-forming materials by the wedge. After manufacture, the drug formulation-filled capsules are dried in the presence of forced air, and a semipermeable lamina encapsulated thereto.

The reciprocating die process produces capsules by leading two films of capsule lamina-forming material between a set of vertical dies. The dies as they close, open, and close perform as a continuous vertical plate forming row after row of pockets across the film. The pockets are filled with an inventive drug formulation, and as the pockets move through the dies, they are sealed, shaped, and cut from the moving film as capsules filled with drug formulation. A semipermeable encapsulating lamina is coated thereon to yield the capsule. The continuous process is a manufacturing system that also uses rotary dies, with the added feature that the process can successfully fill drug in dry powder form into a soft capsule, in addition to encapsulating liquids. The filled capsule of the continuous process is encapsulated with a semipermeable polymeric material to yield the capsule. Procedures for manufacturing soft capsules are disclosed in U.S. Pat. No. 4,627,850 and U.S. Pat. No. 6,419,952.

The dosage forms of the present invention can also be made from an injection-moldable composition by an injection-molding technique. Injection-moldable compositions provided for injection-molding into the semipermeable wall comprise a thermoplastic polymer, or the compositions comprise a mixture of thermoplastic polymers and optional injection-molding ingredients. The thermoplastic polymer that can be used for the present purpose comprise polymers that have a low softening point, for example, below 200° C., preferably within the range of 40° C. to 180° C. The polymers, are preferably synthetic resins, addition polymerized resins, such as polyamides, resins obtained from diepoxides and primary alkanolamines, resins of glycerine and phthalic anhydrides, polymethane, polyvinyl resins, polymer resins with end-positions free or esterified carboxyl or caboxamide groups, for example with acrylic acid, acrylic amide, or acrylic acid esters, polycaprolactone, and its copolymers with dilactide, diglycolide, valerolactone and decalactone, a resin composition comprising polycaprolactone and polyalkylene oxide, and a resin composition comprising polycaprolactone, a polyalkylene oxide such as polyethylene oxide, poly(cellulose) such as poly(hydroxypropylmethylcellulose), poly(hydroxyethylmethylcellulose), and poly(hydroxypropylcellulose). The membrane forming composition can comprise optional membrane-forming ingredients such as polyethylene glycol, talcum, polyvinylalcohol, lactose, or polyvinyl pyrrolidone. The compositions for forming an injection-molding polymer composition can comprise 100% thermoplastic polymer. The composition in another embodiment comprises 10% to 99% of a thermoplastic polymer and 1% to 90% of a different polymer with the total equal to 100%. The invention provides also a thermoplastic polymer composition comprising 1% to 98% of a first thermoplastic polymer, 1% to 90% of a different, second polymer and 1% to 90% of a different, third polymer with all polymers equal to 100%. Representation composition comprises 20% to 90% of thermoplastic polycaprolactone and 10% to 80% of poly(alkylene oxide); a composition comprising 20% to 90% polycaprolactone and 10% to 60% of poly(ethylene oxide) with the ingredients equal to 100%; a composition comprising 10% to 97% of polycaprolactone, 10% to 97% poly(alkylene oxide), and 1% to 97% of poly(ethylene glycol) with all ingredients equal to 100%; a composition comprising 20% to 90% polycaprolactone and 10% to 80% of poly(hydroxypropylcellulose) with all ingredients equal to 100%; and a composition comprising 1% to 90% polycaprolactone, 1% to 90% poly(ethylene oxide), 1% to 90% poly(hydroxypropylcellulose) and 1% to 90% poly(ethylene glycol) with all ingredients equal to 100%. Percent is expressed as weight percent (wt %).

In another embodiment of the invention, a composition for injection-molding to provide a membrane can be prepared by blending a composition comprising a polycaprolactone 63 wt %, polyethylene oxide 27 wt %, and polyethylene glycol 10 wt % in a conventional mixing machine, such as a Moriyama™ Mixer at 65° C. to 95° C., with the ingredients added to the mixer in the following addition sequence, polycaprolactone, polyethylene oxide and polyethylene glycol. In one example, all the ingredients are mixed for 135 minutes at a rotor speed of 10 to 20 rpm. Next, the blend is fed to a Baker Perkins Kneader™ extruder at 80° C. to 90° C., at a pump speed of 10 rpm and a screw speed of 22 rpm, and then cooled to 1 0° C. to 12° C., to reach a uniform temperature. Then, the cooled extruded composition is fed to an Albe Pelletizer, converted into pellets at 250° C., and a length of 5 mm. The pellets next are fed into an injection-molding machine, an Arburg Allrounder™ at 200° F. to 350° C. (93° C. to 177° C.), heated to a molten polymeric composition, and the liquid polymer composition forced into a mold cavity at high pressure and speed until the mold is filled and the composition comprising the polymers are solidified into a preselected shape. The parameters for the injection-molding consists of a band temperature through zone 1 to zone 5 of the barrel of 195° F. (91° C.) to 375° F., (191° C.), an injection-molding pressure of 1818 bar, a speed of 55 cm3/s, and a mold temperature of 75° C. The injection-molding compositions and injection-molding procedures are disclosed in U.S. Pat. No. 5,614,578.

Alternatively, the capsule can be made conveniently in two parts, with one part (the “cap”) slipping over and capping the other part (the “body”) as long as the capsule is deformable under the forces exerted by the expandable layer and seals to prevent leakage of the liquid drug formulation from between the telescoping portions of the body and cap. The two parts completely surround and capsulate the internal lumen that contains the liquid drug formulation, which can contain useful additives. The two parts can be fitted together after the body is filled with a preselected formulation. The assembly can be done by slipping or telescoping the cap section over the body section, and sealing the cap and body, thereby completely surrounding and encapsulating the formulation of drug.

Soft capsules typically have a wall thickness that is greater than the wall thickness of hard capsules. For example, soft capsules can, for example, have a wall thickness on the order of 1040 mils, about 20 mils being typical, whereas hard capsules can, for example, have a wall thickness on the order of 2-6 mils, about 4 mils being typical.

In one embodiment of the dosage system, a soft capsule can be of single unit construction and can be surrounded by an unsymmetrical hydro-activated layer as the expandable layer. The expandable layer will generally be unsymmetrical and have a thicker portion remote from the exit orifice. As the hydro-activated layer imbibes and/or absorbs external fluid, it expands and applies a push pressure against the wall of capsule and optional barrier layer and forces drug formulation through the exit orifice. The presence of an unsymmetrical layer functions to assure that the maximum dose of drug is delivered from the dosage form, as the thicker section of layer distant from passageway swells and moves towards the orifice.

In yet another configuration, the expandable layer can be formed in discrete sections that do not entirely encompass an optionally barrier layer-coated capsule. The expandable layer can be a single element that is formed to fit the shape of the capsule at the area of contact. The expandable layer can be fabricated conveniently by tableting to form the concave surface that is complementary to the external surface of the barrier-coated capsule. Appropriate tooling such as a convex punch in a conventional tableting press can provide the necessary complementary shape for the expandable layer. In this case, the expandable layer is granulated and compressed, rather than formed as a coating. The methods of formation of an expandable layer by tableting are well known, having been described, for example in U.S. Pat. Nos. 4,915,949; 5,126,142; 5,660,861; 5,633,011; 5,190,765; 5,252,338; 5,620,705; 4,931,285; 5,006,346; 5,024,842; and 5,160,743.

In some embodiments, a barrier layer can be first coated onto the capsule and then the tableted, expandable layer is attached to the barrier-coated capsule with a biologically compatible adhesive. Suitable adhesives include, for example, starch paste, aqueous gelatin solution, aqueous gelatin/glycerin solution, acrylate-vinylacetate based adhesives such as Duro-Tak adhesives (National Starch and Chemical Company), aqueous solutions of water soluble hydrophilic polymers such as hydroxypropyl methyl cellulose, hydroxymethyl cellulose, hydroxyethyl cellulose, and the like. That intermediate dosage form can be then coated with a semipermeable layer. The exit orifice is formed in the side or end of the capsule opposite the expandable layer section. As the expandable layer imbibes fluid, it will swell. Since it is constrained by the semipermeable layer, as it expands it will compress the barrier-coated capsule and express the liquid drug formulation from the interior of the capsule into the environment of use.

The hard capsules are typically composed of two parts, a cap and a body, which are fitted together after the larger body is filled with a preselected appropriate formulation. This can be done by slipping or telescoping the cap section over the body section, thus completely surrounding and encapsulating the drug formulation. Hard capsules can be made, for example, by dipping stainless steel molds into a bath containing a solution of a capsule lamina-forming material to coat the mold with the material. Then, the molds are withdrawn, cooled, and dried in a current of air. The capsule is stripped from the mold and trimmed to yield a lamina member with an internal lumen. The engaging cap that telescopically caps the formulation receiving body is made in a similar manner. Then, the closed and filled capsule can be encapsulated with a semipermeable lamina. The semipermeable lamina can be applied to capsule parts before or after parts and are joined into the final capsule. In another embodiment, the hard capsules can be made with each part having matched locking rings near their opened end that permit joining and locking together the overlapping cap and body after filling with formulation. In this embodiment, a pair of matched locking rings are formed into the cap portion and the body portion, and these rings provide the locking means for securely holding together the capsule. The capsule can be manually filled with the drug formulation, or they can be machine filled with the drug formulation. In the final manufacture, the hard capsule is encapsulated with a semipermeable lamina permeable to the passage of fluid and substantially impermeable to the passage of drug. Methods of forming hard cap dosage forms are described in U.S. Pat. No. 6,174,547, U.S. Pat. Nos. 6,596,314, 6,419,952, and 6,174,547.

The hard and soft capsules can comprise, for example, gelatin; gelatin having a viscosity of 15 to 30 millipoises and a bloom strength up to 150 grams; gelatin having a bloom value of 160 to 250; a composition comprising gelatin, glycerine, water and titanium dioxide; a composition comprising gelatin, erythrosin, iron oxide and titanium dioxide; a composition comprising gelatin, glycerine, sorbitol, potassium sorbate and titanium dioxide; a composition comprising gelatin, acacia glycerine, and water; and the like. Materials useful for forming capsule wall are known in U.S. Pat. Nos. 4,627,850; and in 4,663,148. Alternatively, the capsules can be made out of materials other than gelatin (see for example, products made by BioProgres plc).

The capsules typically can be provided, for example, in sizes from about 3 to about 22 minims (1 minim being equal to 0.0616 ml) and in shapes of oval, oblong or others. They can be provided in standard shape and various standard sizes, conventionally designated as (000), (00), (0), (1), (2), (3), (4), and (5). The largest number corresponds to the smallest size. Non-standard shapes can be used as well. In either case of soft capsule or hard capsule, non-conventional shapes and sizes can be provided if required for a particular application.

The osmotic devices of the present invention may comprise a semipermeable wall permeable to the passage of exterior biological fluid and substantially impermeable to the passage of compound formulation. The selectively permeable compositions used for forming the wall are essentially non-erodible and they are insoluble in biological fluids during the life of the osmotic system. The semipermeable wall comprises a composition that does not adversely affect the host, the compound formulation, an osmopolymer, osmagent and the like. Materials useful in the formation of a semipermeable wall are disclosed elsewhere herein.

The semipermeable wall can also comprise a flux regulating agent. Materials useful flux regulating agents are disclosed elsewhere herein. Other materials that can be used to form the semipermeable wall for imparting flexibility and elongation properties to the semipermeable wall are also disclosed elsewhere herein.

The semipermeable wall surrounds and forms a compartment containing a one or a plurality of layers, one of which is an expandable layer which in some embodiments, can contain osmotic agents. The composition of such expandable layers is disclosed elsewhere herein.

In certain solid and liquid embodiments, the dosage forms further can comprise a barrier layer. The barrier layer in certain embodiments is deformable under the pressure exerted by the expandable layer and will be impermeable (or less permeable) to fluids and materials that can be present in the expandable layer, the liquid drug formulation and in the environment of use, during delivery of the drug formulation. A certain degree of permeability of the barrier layer can be permitted if the delivery rate of the drug formulation is not detrimentally effected. However, it is preferred that barrier layer not completely transport through it fluids and materials in the dosage form and the environment of use during the period of delivery of the drug. The barrier layer can be deformable under forces applied by expandable layer so as to permit compression of capsule to force the liquid drug formulation from the exit orifice. In some embodiments, the barrier layer will be deformable to such an extent that it create a seal between the expandable layer and the semipermeable layer in the area where the exit orifice is formed. In that manner, the barrier layer will deform or flow to a limited extent to seal the initially, exposed areas of the expandable layer and the semipermeable layer when the exit orifice is being formed, such as by drilling or the like, or during the initial stages of operation. When sealed, the only avenue for liquid permeation into the expandable layer is through the semipermeable layer, and there is no back-flow of fluid into the expandable layer through the exit orifice.

Suitable materials for forming the barrier layer can include, for example, polyethylene, polystyrene, ethylene-vinyl acetate copolymers, polycaprolactone and Hytrel™ polyester elastomers (Du Pont), cellulose acetate, cellulose acetate pseudolatex (such as described in U.S. Pat. No. 5,024,842), cellulose acetate propionate, cellulose acetate butyrate, ethyl cellulose, ethyl cellulose pseudolatex (such as Surelease™ as supplied by 10 Colorcon, West Point, Pa. or Aquacoat™ as supplied by FMC Corporation, Philadelphia, Pa.), nitrocellulose, polylactic acid, poly-glycolic acid, polylactide glycolide copolymers, collagen, polyvinyl alcohol, polyvinyl acetate, polyethylene vinylacetate, polyethylene teraphthalate, polybutadiene styrene, polyisobutylene, polyisobutylene isoprene copolymer, polyvinyl chloride, polyvinylidene chloride-vinyl chloride copolymer, copolymers of acrylic acid and methacrylic acid esters, copolymers of methylmethacrylate and ethylacrylate, latex of acrylate esters (such as Eudragit™ supplied by RohmPharma, Darmstaat, Germany), polypropylene, copolymers of propylene oxide and ethylene oxide, propylene oxide ethylene oxide block copolymers, ethylenevinyl alcohol copolymer, polysulfone, ethylene vinylalcohol copolymer, polyxylylenes, polyalkoxysilanes, polydimethyl siloxane, polyethylene glycol-silicone elastomers, electromagnetic irradiation crosslinked acrylics, silicones, or polyesters, thermally crosslinked acrylics, silicones, or polyesters, butadiene-styrene rubber, and blends of the above.

Preferred materials can include cellulose acetate, copolymers of acrylic acid and methacrylic acid esters, copolymers of methylmethacrylate and ethylacrylate, and latex of acrylate esters. Preferred copolymers can include poly (butyl methacrylate), (2-dimethylaminoethyl)methacrylate, methyl methacrylate) 1:2:1, 150,000, sold under the trademark EUDRAGIT E; poly (ethyl acrylate, methyl methacrylate) 2:1, 800,000, sold under the trademark EUDRAGIT NE 30 D; poly (methacrylic acid, methyl methacrylate) 1:1, 135,000, sold under the trademark EUDRAGIT L; poly (methacrylic acid, ethyl acrylate) 1:1, 250,000, sold under the trademark EUDRAGIT L; poly (methacrylic acid, methyl methacrylate) 1:2, 135,000, sold under the trademark EUDRAGIT S; poly (ethyl acrylate, methyl methacrylate, trimethylammonioethyl methacrylate chloride) 1:2:0.2, 150,000, sold under the trademark EUDRAGIT RL; poly (ethyl acrylate, methyl methacrylate, trimethylammonioethyl methacrylate chloride) 1:2:0.1, 150,000, sold as EUDRAGIT RS. In each case, the ratio x:y:z indicates the molar proportions of the monomer units and the last number is the number average molecular weight of the polymer. Especially preferred are cellulose acetate containing plasticizers such as acetyl tributyl citrate and ethylacrylate methylmethylacrylate copolymers such as Eudragit NE.

The foregoing materials for use as the barrier layer can be formulated with plasticizers to make the barrier layer suitably deformable such that the force exerted by the expandable layer will collapse the compartment formed by the barrier layer to dispense the liquid drug formulation. Examples of typical plasticizers are as follows: polyhydric alcohols, triacetin, polyethylene glycol, glycerol, propylene glycol, acetate esters, glycerol triacetate, triethyl citrate, acetyl triethyl citrate, glycerides, acetylated monoglycerides, oils, mineral oil, castor oil and the like. The plasticizers can be blended into the material in amounts of 10-50 weight percent based on the weight of the material.

The various layers forming the barrier layer, expandable layer and semipermeable layer can be applied by conventional coating methods such as described in U.S. Pat. No. 5,324,280. While the barrier layer, expandable layer and semipermeable wall have been illustrated and described for convenience as single layers, each of those layers can be composites of several layers. For example, for particular applications it may be desirable to coat the capsule with a first layer of material that facilitates coating of a second layer having the permeability characteristics of the barrier layer. In that instance, the first and second layers comprise the barrier layer. Similar considerations would apply to the semipermeable layer and the expandable layer.

The exit orifice can be formed by mechanical drilling, laser drilling, eroding an erodible element, extracting, dissolving, bursting, or leaching a passageway former from the composite wall. The exit orifice can be a pore formed by leaching sorbitol, lactose or the like from a wall or layer as disclosed in U.S. Pat. No. 4,200,098. This patent discloses pores of controlled-size porosity formed by dissolving, extracting, or leaching a material from a wall, such as sorbitol from cellulose acetate. A preferred form of laser drilling is the use of a pulsed laser that incrementally removes material from the composite wall to the desired depth to form the exit orifice.

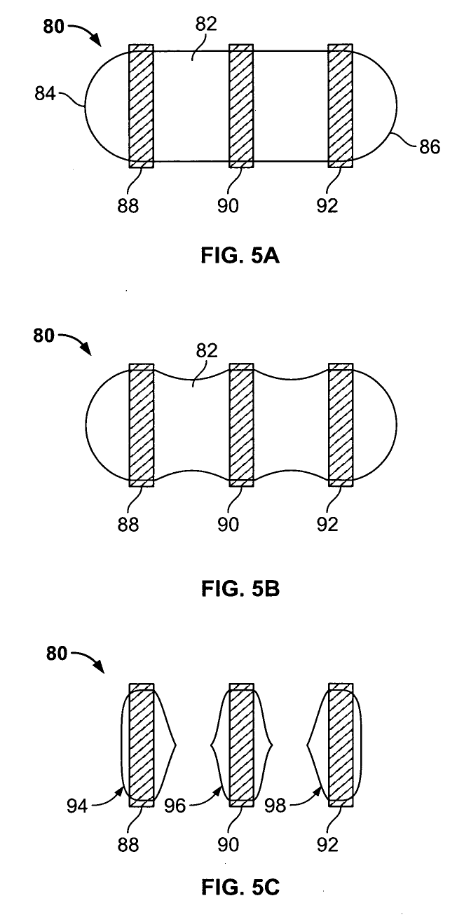

FIGS. 5A-5C illustrate another exemplary dosage form, known in the art and described in U.S. Pat. Nos. 5,534,263; 5,667,804; and 6,020,000. Briefly, a cross-sectional view of a dosage form 80 is shown prior to ingestion into the gastrointestinal tract in FIG. 5A. The dosage form is comprised of a cylindrically shaped matrix 82 comprising a compound of Formula (I) or Formula (II). Ends 84, 86 of matrix 82 are preferably rounded and convex in shape in order to ensure ease of ingestion. Bands 88, 90, and 92 concentrically surround the cylindrical matrix and are formed of a material that is relatively insoluble in an aqueous environment. Suitable materials are set forth in the patents noted above and elsewhere herein.

After ingestion of dosage form 80, regions of matrix 82 between bands 88, 90, 92 begin to erode, as illustrated in FIG. 5B. Erosion of the matrix initiates release of the compound of Formula (I) or Formula (II) into the fluidic environment of the G.I. tract. As the dosage form continues transit through the G.I. tract, the matrix continues to erode, as illustrated in FIG. 5C. Here, erosion of the matrix has progressed to such an extent that the dosage form breaks into three pieces, 94, 96, 98. Erosion will continue until the matrix portions of each of the pieces have completely eroded. Bands 94, 96, 98 will thereafter be expelled from the G.I. tract.

In an embodiment, the inventive sustained release dosage forms comprise gastric retention dosage forms. U.S. Pat. No. 5,007,790 to Shell, granted Apr. 16, 1991 and entitled “Sustained-release oral drug dosage form” (“Shell”) discloses a gastric retention dosage form useful in the practice of this invention. Shell discloses sustained-release oral drug dosage forms that release drug in solution at a rate controlled by the solubility of the drug. The dosage form comprises a tablet or capsule which comprises a plurality of particles of a dispersion of a limited solubility drug in a hydrophilic, water-swellable, crosslinked polymer that maintains its physical integrity over the dosing lifetime but thereafter rapidly dissolves. Once ingested, the particles swell to promote gastric retention and permit the gastric fluid to penetrate the particles, dissolve drug and leach it from the particles. One or more compounds of Formula (I) or Formula (II) may be incorporated into such a gastric retention dosage form, or others known in the art, in the practice of this invention.

A preferred embodiment of the osmotic sustained release dosage form is generally disclosed in U.S. Pat. Nos. 6,368,626 and 6,855,334, among others, and is known as a OROS® Push-Stick™ drug delivery system. An advantage of such systems is that they can achieve a much higher drug loading than prior osmotic sustained release dosage forms.

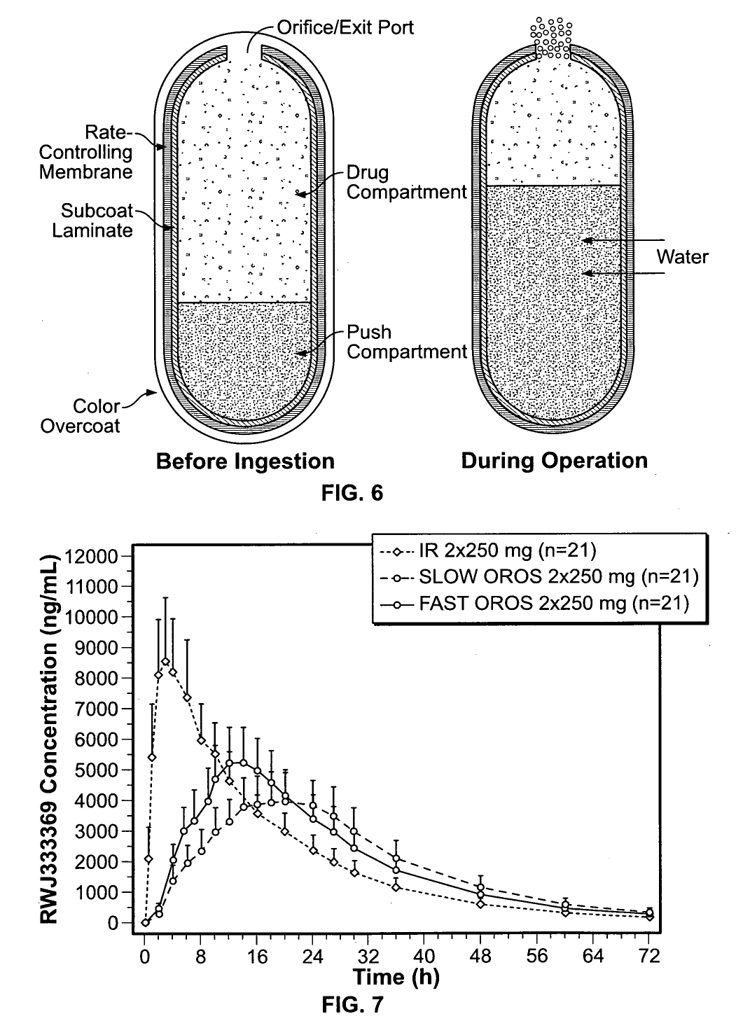

A preferred embodiment of a dosage form of this invention having the Push-Stick™ configuration is illustrated in FIG. 6 prior to its administration to a subject, during operation and after delivery of the active agent. The dosage form comprises a wall defining a cavity and an exit orifice. Within the cavity and remote from the exit orifice is a push displacement layer, and a drug layer is located within cavity adjacent the exit orifice. A flow-promoting layer extends at least between the drug layer and the inner surface of the wall, and can extend between the inner surface of the wall and the push displacement layer. The dosage form is at high drug loading in the drug layer based on the overall weight of the drug layer. The drug is exposed to the environment of use as an erodible composition.

In a preferred embodiment, the oral dosage form comprises a tablet comprising approximately 250 mg of a compound of Fomrula (I) or Formula (II), butylated hydroxytoluene, carnauba wax, cellulose acetate, croscarmellose sodium, hydroxyethyl cellulose, hydroxypropyl cellulose, magnesium stearate, white OPADRY filmc-coating, poloxamer 188, polyethylene glycol, polyethylene oxide, povidone, red and yellow iron oxide, sodium chloride, and stearic acid. The tablet dimensions may be approximately 13.7 mm (length) by 6.7 mm (diameter).

Other approaches to achieving sustained release of drugs from oral dosage forms are known in the art. For example, diffusion systems such as reservoir devices and matrix devices, dissolution systems such as encapsulated dissolution systems (including, for example, “tiny time pills”) and matrix dissolution systems, combination diffusion/dissolution systems and ion-exchange resin systems are known and are disclosed in Remington's Pharmaceutical Sciences, 1990 ed., pp. 1682-1685. Dosage forms that operate in accord with these other approaches are encompassed by the scope of the disclosure herein to the extent that the drug release characteristics and/or the blood plasma concentration characteristics as recited herein and in the claims describe those dosage forms either literally or equivalently.

Examples of dosage forms that may be useful in the practice of the present invention include U.S. Pat. Nos. 5,871,778 and 5,656,299, which disclose sustained microsphere formulations having almost zero order rate of release of active component when administered to a patient. Additionally, U.S. Pat. Nos. 5,654,008; 5,650,173; 5,770,231; 6,077,843; 6,368,632; and 5,965,168 disclose sustained-release microparticle compositions, which may be useful in the practice of this invention.

It will be appreciated the dosage forms described herein, particularly in FIGS. 1-6 are merely exemplary of a variety of dosage forms designed for and capable of achieving administration of the inventive substance(s). Those of skill in the pharmaceutical arts can identify other dosage forms that would be suitable.

Methods of Use

The inventive methods, compositions, and dosage forms are useful in treating a variety of indications that are treatable using compounds of Formula (I) or Formula (II). In an aspect, the invention provides a method for treating an indication, such as a disease or disorder, in a patient by administering an inventive composition or dosage form that comprises one or more compounds of Formula (I) or Formula (II). In an embodiment, a composition or dosage form comprising one or more compounds of Formula (I) or Formula (II) is administered to the patient via oral administration. The dose administered is generally adjusted in accord with the age, weight, and condition of the patient, taking into consideration the dosage form and the desired result. Inventive dosage forms may comprise one or more compounds of Formula (I) or Formula (II), or pharmacologically active metabolites in combination.

It will be readily apparent to those skilled in the art that any dose or frequency of administration that provides the therapeutic or prophylactic effect described herein is suitable for use in the present invention. Dosage regimens may be varied depending upon the requirement of the subjects (including factors associated with the particular subject being treated, including subject age, weight and diet, strength of the preparation, the advancement of the disease condition and the mode and time of administration) and the use of a particular compound of Formula (I) or Formula (II) or pharmaceutical composition thereof or a pharmaceutically acceptable salt thereof. Optimal dosages to be administered may be readily determined by those skilled in the art and will result in the need to adjust the dose to an appropriate therapeutic or prophylactic level. The use of either daily administration or post-periodic dosing may be employed.

In an embodiment, dosage forms according to the invention comprise an amount of the one or more compounds of Formula (I) or Formula (II) ranging from about 20 mg to about 5000 mg, preferably from about 50 mg to about 3000 mg, and more preferably from about 100 mg to about 2000 mg.

While there has been described and pointed out features and advantages of the invention, as applied to present embodiments, those skilled in the medical art will appreciate that various modifications, changes, additions, and omissions in the method described in the specification can be made without departing from the spirit of the invention.

The present invention is not to be limited in terms of the particular embodiments described in this application, which are intended as single illustrations of individual aspects of the invention. Many modifications and variations of this invention can be made without departing from its spirit and scope, as will be apparent to those skilled in the art. Functionally equivalent methods within the scope of the invention, in addition to those enumerated herein, will be apparent to those skilled in the art from the foregoing description. Such modifications and variations are intended to fall within the scope of the appended claims. The present invention is to be limited only by the terms of the appended claims, along with the full scope of equivalents to which such claims are entitled.

The following Examples are meant to be illustrative of the claimed invention, and not limiting in any way.

### EXAMPLES

### Example 1

### (Fast OROS) RWJ-333369 Capsule Shaped Tablet, Bilayer 250 MG System

A dosage form adapted, designed and shaped as an osmotic drug delivery device was manufactured as follows:

A drug granulation was prepared as follows: 2100 g of micronized RWJ-333369, 412.2 g of polyethylene oxide N-80 with average molecular weight of 200,000, 240 g of Poloxamer 188,120 g of povidone (K29-32), and 90 g of croscarmellose sodium were added to a granulator bowl. The dry components were dry-blended, and Ethyl Alcohol SDA 3A, anhydrous, was slowly metered into the mixing bowl. The rate of solvent addition and mixer speed was adjusted as necessary to achieve an acceptable granulation. The the wet granulation was next manually sized using a 16-mesh screen, and then dried under ambient conditions until the average Loss on Drying (LOD) fell within the acceptable range (0.5-1.5%). The dried granules were then sized using 16-mesh screen.

Next the sized granulation was charged into a V-Blender, and 30 g stearic acid and 0.3 g milled BHT were individually sieved through a 40-mesh screen, charged to the V-Blender and mixed. A final moisture content determination by LOD fell within the acceptable range of 0.5-1.5%.

Next, a push layer granulation was prepared. A binder solution was prepared by dispensing 155.8 kg of purified water into a solution tank. Then 27.5 kg povidone (K29-32) was added, and the solution mixed to ensure freedom of solids. Using a 6-mesh screen, approximately half of 289.35 kg of polyethylene oxide of approximately 7,000,000 MW was charged directly into a fluid bed granulator. Next, 0.45 kg of Ferric Oxide Red, 1.35 kg of Ferric Oxide Yellow, and 135 kg of sodium chloride were charged into the fluid bed granulator. Finally, using a 6-mesh screen, the remaining amount of polyethylene oxide was charged into the granulator. The fluid bed granulator was then operated, and the required amount of binder solution sprayed into the bowl. The drying cycle was terminated when proper moisture content was achieved. [Moisture content (LOD at 75° C., Range: <1.0%)]. The dried granulation was sized by passing through a 7-mesh screen and then was charged into a tote.

0.225 kg of milled BHT was sieved passing through a 40-mesh screen. The BHT was manually dispersed into the milled granulation in the tote and blended on a tote tumbler. Next, 1.125 kg of stearic acid NF was sieved through a 40-mesh screen, added to granulation in the tote and blended in the tote tumbler.

Cores were manufactured using a Korsch tablet press, using the following settings:

| | |
|---|---|
| Number of stations | 6 |
| Punch/Die set | ¼″, deep concave |
| First layer compression force (N) | 175 |
| First layer weight (mg/core) | 357 |
| Final compression force (N) | 5000 |
| Compression speed (rpm) | 15 |
| Total bilayer core weight (mg/core) | 476 |
| Thickness (mm) | 13 |

The cores were sub-coated as follows: 18400 g of ethyl alcohol SDA 3A anhydrous was weighed and charged into a mixing vessel which was then activated. 480 g of povidone (K29-32) was charged into the mixing vessel and mixed until a clear solution resulted. 1120 g of hydroxypropyl cellulose was then charged into the mixing vessel and mixed until a clear solution resulted. The solution was sprayed onto uncoated active and uncoated lactose filler cores in a pan coating chamber until the target wet subcoat weight of 25 mg was reached. The cores were then removed from the chamber. Note: The load in the pan included 5/16″ round lactose filler cores along with the active RWJ 333369 cores. The lactose cores were used as filler cores to achieve required load capacity for pan coating.

The membrane coating was applied as follows: 47300 g of acetone was charged into a mixing vessel and the mixer turned on. 238 g of water, 500 grams of polaxmer 188, and 2000 g of cellulose acetate (398-10) were then added to the mixing vessel and mixed until a clear solution resulted. The membrane solution was applied onto active and lactose filler cores using a pan coater until the target wet membrane weight of 35 mg was reached. The cores were then removed from the chamber. Note: The load in the pan included 5/16″ round lactose filler cores along with the active RWJ 333369 cores. The lactose cores were used as filler cores to achieve required load capacity for pan coating.

The coated active cores then had a 4.5 mm target diameter orifice drilled on the drug layer end of the system using a LCT Laser and ⅜″ carriers. The drilled tablets were placed on perforated stainless steel trays, which were inserted into a relative humidity oven set at 40° C. and 40% relative humidity and allowed to dry for at least 72 hours. The drilled tablets were allowed to dry for at least an additional 4 hours at 40° C. at ambient RH conditions.

### Example 2

### (Slow OROS) RWJ-333369 Capsule Shaped Tablet Bilayer 250 MG System

A drug granulation was prepared as follows: 2100 g of micronized RWJ-333369, 412.2 g of polyethylene oxide N-80 with average molecular weight of 200,000, 240 g of Poloxamer 188,120 g of povidone (K29-32), and 90 g of croscarmellose sodium were added to a granulator bowl. The dry components were dry-blended, and Ethyl Alcohol SDA 3A, anhydrous, was slowly metered into the mixing bowl. The rate of solvent addition and mixer speed was adjusted as necessary to achieve an acceptable granulation. The the wet granulation was next manually sized using a 16-mesh screen, and then dried under ambient conditions until the average Loss on Drying (LOD) fell within the acceptable range (0.5-1.5%). The dried granules were then sized using 16-mesh screen.

Next the sized granulation was charged into a V-Blender, and 30 g stearic acid and 0.3 g milled BHT were individually sieved through a 40-mesh screen, charged to the V-Blender and mixed. A final moisture content determination by LOD fell within the acceptable range of 0.5-1.5%.

Next, a push layer granulation was prepared. A binder solution was prepared by dispensing 155.8 kg of purified water into a solution tank. Then 27.5 kg povidone (K29-32) was added, and the solution mixed to ensure freedom of solids. Using a 6-mesh screen, approximately half of 289.35 kg of polyethylene oxide of approximately 7,000,000 MW was charged directly into a fluid bed granulator. Next, 0.45 kg of Ferric Oxide Red, 1.35 kg of Ferric Oxide Yellow, and 135 kg of sodium chloride were charged into the fluid bed granulator. Finally, using a 6-mesh screen, the remaining amount of polyethylene oxide was charged into the granulator. The fluid bed granulator was then operated, and the required amount of binder solution sprayed into the bowl. The drying cycle was terminated when proper moisture content was achieved. [Moisture content (LOD at 75° C., Range:≦1.0%)]. The dried granulation was sized by passing through a 7-mesh screen and then was charged into a tote.

0.225 kg of milled BHT was sieved passing through a 40-mesh screen. The BHT was manually dispersed into the milled granulation in the tote and blended on a tote tumbler. Next, 1.125 kg of stearic acid was sieved through a 40-mesh screen, added to granulation in the tote and blended in the tote tumbler.

Cores were manufactured using a Korsch tablet press, using the following settings:

| | |
|---|---|
| Number of stations | 6 |
| Punch/Die set | ¼″, deep concave |
| First layer compression force (N) | 175 |
| First layer weight (mg/core) | 357 |
| Final compression force (N) | 5000 |
| Compression speed (rpm) | 15 |
| Total bilayer core weight (mg/core) | 476 |
| Thickness (mm) | 13 |

The cores were sub-coated as follows: 18400 g of ethyl alcohol SDA 3A, anhydrous, was weighed and charged into a mixing vessel which was then activated. 480 g of povidone (K29-32) was charged into the mixing vessel and mixed until a clear solution resulted. 1120 g of hydroxypropyl cellulose was then charged into the mixing vessel and mixed until a clear solution resulted. The solution was sprayed onto uncoated active and uncoated lactose filler cores in a pan coating chamber until the target wet subcoat weight of 25 mg was reached. The cores were then removed from the chamber. Note: The load in the pan included 5/16″ round lactose filler cores along with the active RWJ 333369 cores. The lactose cores were used as filler cores to achieve required load capacity for pan coating.

The membrane coating was applied as follows: 47300 g of acetone was charged into a mixing vessel and the mixer turned on. 238 g of water, 375 grams of polaxmer 188, and 2125 g of cellulose acetate (398-10) were then added to the mixing vessel and mixed until a clear solution resulted. The membrane solution was applied onto active and lactose filler cores using a pan coater until the target wet membrane weight of 39 mg was reached. The cores were then removed from the chamber. Note: The load in the pan included 5/16″ round lactose filler cores along with the active RWJ 333369 cores. The lactose cores were used as filler cores to achieve required load capacity for pan coating.

The coated active cores then had a 4.5 mm target diameter orifice drilled on the drug layer end of the system using a LCT Laser and ⅜″ carriers. The drilled tablets were placed on perforated stainless steel trays, which were inserted into a relative humidity oven set at 40° C. and 40% relative humidity and allowed to dry for at least 72 hours. The drilled tablets were allowed to dry for at least an additional 4 hours at 40° C. at ambient RH conditions.

### Example 3

### Evaluation of OROS RWJ33369 and IR RWJ33369 Pharmacokinetics

This study investigated the pharmacokinetics of 2 different formulations of OROS®( RWJ333369) and compared with IR RWJ333369. This was a single-center, single-dose, open-label, three-treatment, three-period, six-sequence crossover study. Each subject received the following treatments in the fasted state in a random manner.

Treatment A—Single dose of two 250 mg capsules of IR RWJ333369

Treatment B—Single dose of two 250 mg tablets of SLOW OROS® RWJ333369 (made according to Example 2)

Treatment C—Single dose of two 250 mg tablets of FAST OROS® RWJ333369 (made according to Example 1)

Twenty-four healthy males and females were enrolled and 21 subjects received all three study treatments. FAST OROS(D and SLOW OROS® were designed to deliver the doses in approximately 9 hours and 16 hours, respectively. There was a 6- to 14-day washout period between treatments, which began 24 hours from dosing in each treatment period. During each treatment, blood samples were collected from each subject to determine plasma RWJ333369 concentrations. Samples were collected at:

IR: 0 (pre-dose), 0.5, 1, 2, 3, 4, 6, 8, 10, 12, 16, 20, 24, 27, 30, 36, 48, 60, and 72 hours post dosing.

SLOW OROS®: 0 (predose), 2, 4, 6, 8, 10, 12, 14, 16, 18, 20, 24, 27, 30, 36, 48, 60, and 72 hours post dosing.

FAST OROS®: 0 (predose), 2, 4, 5.5, 7, 9, 10, 12, 14, 16, 18, 20, 24, 27, 30, 36, 48, 60, and 72 hours post dosing.

PK parameters AUCₜ, AUC_{inf}, Cₘₐₓ, Tₘₐₓ, and t_{1/2} were calculated for RWJ333369 for each treatment and subject as described in Example 1.

FIG. 7 illustrates the mean RWJ333369 plasma concentration profile. Pharmacokinetic parameters as well as ratios and 90% CIs are summarized in Table 1.

The SLOW OROS® treatments resulted in a lower Cₘₐₓ and provided later peaks (Tₘₐₓ) compared with IR RWJ333369. FAST OROS® treatment also resulted in a lower Cₘₐₓ and provide later peaks (Tₘₐₓ) compared with the IR RWJ333369, but to a lesser degree than the SLOW OROS® treatments. SLOW and FAST OROS® RWJ333369 had a mean Cₘₐₓ approximately 46% and 61% of that of the IR formulation. Mean half-life for RWJ333369 values were similar among the three treatments (approximately 12 hours), while tₘₐₓ was longer for SLOW and FAST OROS® RWJ333369 (approximately 20 and 14 hours, respectively) than for the IR (approximately 3 hours) formulation.

Mean bioavailability estimated for FAST OROS® (RWJ333369) and SLOW OROS® (RWJ333369) in the fasted state was approximately 94% and 91%, respectively, relative to IR RWJ333369. The results of the ANOVA and 90% confidence intervals are also presented in Table 1.

TABLE 1
Pharmacokinetic Values Following Three RWJ333369 Treatments

| | IR 2 × 250 mg | | SLOW OROS | | FAST OROS | |
|---|---|---|---|---|---|---|
| N = 21 | capsules | | 2 × 250 mg | | 2 × 250 mg | |
| RWJ333369 Concentration | | | Mean (SD) | | | |
| Cₘₐₓ (ng/mL) | 9178 | (2207) | 4242 | (836) | 5635 | (1135) |
| Tₘₐₓ (h) | 2.95 | (1.02) | 20.0 | (3.53) | 13.6 | (2.25) |
| T_{1/2} (h) | 11.0 | (1.56) | 12.0 | (1.73) | 11.6 | (1.69) |
| AUCₜ | 153126 | (29613) | 136435 | (31404) | 143442 | (31283) |
| (ng · h/mL) | | | | | | |
| AUC₀₋₇₂ | 153409 | (29385) | 136542 | (31309) | 143587 | (31151) |
| (ng · h/mL) | | | | | | |
| AUC_{inf} | 155652 | (30180) | 141542 | (33174) | 147463 | (32620) |
| (ng · h/mL) | | | | | | |

| | | | | | |
|---|---|---|---|---|---|
| Bioavailability | Reference | 91.0 | (11.9) | 94.7 | (9.8) |
| ANOVA | | | | | |

| | | | |
|---|---|---|---|
| AUC_{inf} (vs IR) | Reference | 90.6% | 93.8% |
| Ratio | | (81.6, 100.7) | (84.5, 104.1) |
| (90% CI)^{a} | | | |

Table 2 provides the comparison of side effects that are possibly or probably related to study drug among the three treatments over 72 h post dosing period. Both FAST and SLOW OROS® RWJ333369 had a more favorable adverse event profile: of the 21 subjects, 16 had dizziness after receiving the IR formulation, but only 2 had this event after receiving both Slow and Fast OROS® RWJ333369. The incidence of dizziness was lower in the Slow (10%) and Fast (9%) OROS® treatments compared to the incidence in the IR treatment (67%). The onset of dizziness and euphoria with IR treatment all occurred within 0.1 to 2.2 hr post dosing and lasted for most subjects up to about 4 hours post dosing. With Slow or Fast OROS treatments, the small number of dizziness reported (2 subjects reported dizziness in both treatments) occurred randomly at different times ranging from 0.2 to 5.8 h hr post dosing. Among three of the 4 incidences, the duration of dizziness was relatively short (1-4 h). Duration of the fourth incidence was about 11 h.

TABLE 2
Adverse Event (Possibly or Probably Related to Study Drug)
Incidence^{a}

| | | FAST OROS | Slow OROS |
|---|---|---|---|
| | IR 2 × 250 mg | 2 × 250 mg | 2 × 250 mg |
| | capsules (n = 24) | (n = 23) | (n = 21) |
| AE | No. (%) | No. (%) | No. (%) |
| Headache | 5 (20.8) | 2 (8.7) | 2 (9.5) |
| Dizziness | 16 (66.7) | 2 (8.7) | 2 (9.5) |
| Euphoria | 2 (8.3) | 0 (0.0) | 0 (0.0) |
| Nausea | 2 (8.3) | 0 (0.0) | 1 (4.8) |
| Fatigue | 2 (8.3) | 1 (4.3) | 0 (0.0) |

^{a}Reported for at least 2 subjects in any given treatment over 72 h.

### Example 4

### Liquid Dosage Form

A hard cap oral osmotic device system for dispensing a compound of Formula (I) or of Formula (II) in the G.I. tract may be prepared as follows:

First, an osmotic push-layer formation is granulated using a Glatt fluid bed granulator (FBG). The composition of the push granules is comprised of 63.67 wt % of polyethylene oxide of 7,000,000 molecular weight, 30.00 wt % sodium chloride, 1.00 wt % ferric oxide, 5.00 wt % hydroxypropylmethylcellulose of 9,200 molecular weight, 0.08 wt % butylated hydroxytoluene and 0.25 wt % magnesium stearate.

Second, the barrier layer granulations are produced using a medium FBG. The composition of barrier-layer granules is comprised of 55 wt % Kollidon, 35 wt % Magnesium Stearate and 10 wt % EMM.

Third, the osmotic push layer granules and barrier layer granules are compressed into a bi-layer tablet with a Multi-layer Korsch press. 350 mg of the osmotic push-layer granules are added and tamped, and then 100 mg of barrier layer granules are added onto and finally compressed under a force of 4500 N into an osmotic/barrier bi-layer tablet.

Fourth, 235 mg of the compound of Formula (I) or of Formula (II) are dissolved into about 211 mg propylene glycol (PG) using sonication at 45° C. for ˜6 h to form a drug-layer composition.

Next, gelatin capsules of a sufficient size are subcoated with Surelease™. This will inhibit water-permeation into the capsulated liquid formulation during system operation. The subcoating is a membrane of ethylcellulose applied in the form of aqueous dispersion. The dispersion contains 25 wt % solids and is diluted to contain 15 wt % solids by adding purified water. The membrane weight of Surelease™ is 17 mg.

Next, a Surelease™ coated gelatin capsule is separated into two segments (body and cap). The drug-layer composition (500 mg) is filled into the capsule body.

Next, the osmotic/barrier tablet is placed in the filled capsule body. Before inserting the engines into the capsules, a layer of sealing solution is applied around the barrier layer of the gelatin-coated bilayer engines. After engine insertion, a layer of banding solution is applied around the diameter at the interface of capsule and engine. This sealing and banding solution are the same, which is made of water/ethanol 50/50 wt %.

Next, the membrane composition comprising 80% cellulose acetate 398-10 and 20% Pluronic F-68 is dissolved in acetone with solid content of 5% in the coating solution. The solution is sprayed onto the pre-coating assemblies in a 12″ LDCS Hi-coater. After membrane coating, the systems are dried in oven at 45° C. for 24. The assemblies are coated with 131 mg of the rate-controlling membrane.

Next, a 30 mil (0.77 mm) exit orifice is drilled at the drug-layer side using a mechanical drill. By adjusting the membrane weight, the release duration of the systems can be controlled.

### Example 5

### Solid Dosage Form

A matrix dosage form according to the present invention is prepared as follows: 247 grams of a compound of Formula (I) or Formula (II), 25 grams of hydroxypropyl methylcellulose having a number average molecular weight of 9,200 grams per mole, and 15 grams of hydroxypropyl methylcellulose having a molecular weight of 242,000 grams per mole, are passed through a screen having a mesh size of 40 wires per inch. The celluloses each have an average hydroxyl content of 8 weight percent and an average methoxyl content of 22 weight percent. The resulting sized powders are tumble mixed. Anhydrous ethyl alcohol is added slowly to the mixed powders with stirring until a dough consistency is produced. The damp mass is then extruded through a 20 mesh screen and air dried overnight. The resulting dried material is re-screened through a 20 mesh screen to form the final granules. 2 grams of the tableting lubricant, magnesium stearate, which are sized through an 80 mesh screen, are then tumbled into the granules.

663 mg of the resulting granulation is placed in a die cavity having an inside diameter of 9/32 inch and compressed with deep concave punch tooling using a pressure head of 2 tons. This forms a longitudinal capsule core having an overall length, including the rounded ends, of 0.691 inch. The cylindrical body of the capsule, from tablet land to tablet land, spans a distance of 12 mm.

### Example 6

### Solid Dosage Form with Modified Release Characteristics

Solid dosages forms according to Example 5 are provided. Next, rings of polyethylene having an inside diameter of 9/32 inch, a wall thickness of 0.013 inch, and a width of 2 mm are then fabricated. These rings, or bands, are press fitted onto the core to complete the dosage form.

### Example 7

### Gastric Retention Dosage Form

A dosage form according to the disclosure in U.S. Pat. No. 6,548,083 to Wong, et al., granted Apr. 15, 2003, entitled “Prolonged release active agent dosage form adapted for gastric retention”, is prepared with a compound of Formula (I) or Formula (II).

Eighteen grams of a compound of Formula (I) or Formula (II), and 3.6 grams of a gel-forming polymer polyethylene oxide, having a number average molecular weight of approximately 8 million grams per mole, are separately screened through a mesh having 40 wires per inch. The polyethylene oxide is supplied under the trade name Polyox.RTM. grade 308 as manufactured by Union Carbide Corporation, Danbury, Conn. The sized active agent and polymer are dry mixed. Then, 8.25 grams of a hydroattractant water-insoluble polymer, hydroxypropyl cellulose having a hydroxypropyl content of 10-13 weight percent and an average fiber particle size of 50 microns, is sieved through the 40-mesh screen and blended into the mixture. The hydroxypropyl cellulose is supplied as Low-Substituted Hydroxypropyl Cellulose grade 11 as manufactured by Shin-Etsu Chemical Company, Ltd., Tokyo, Japan. Anhydrous ethyl alcohol, specially denatured formula 3A, i.e., ethanol denatured with 5 volume percent methanol, is added to the mixture with stirring until a uniformly damp mass formed. This damp mass is extruded with pressure through a screen having 20 wires per inch. The extrudate is then allowed to air dry at room temperature overnight. After drying, the resulting extrudate is passed again through the 20-mesh sieve, forming granules. 0.15 Grams of the tableting lubricant, magnesium stearate, are passed through a sieve having 60 wires per inch. The sized 60-mesh lubricant is then tumbled into the granules to produce the finished granulation.

Portions of the resulting granulation are weighed and compacted with caplet-shaped tooling on a Carver press at pressure head of 1.5 tons. Each tablet weighs approximately 1042 mg and contains approximately 625 mg of the active agent. The shape of the tablet has approximately cylindrical proportions. The diameter is approximately 7.6 millimeters (mm) and the length was approximately 22 mm.

A tube of polyolefin material having an outside diameter of 7.7 mm and having a wall thickness of 0.25 mm is sliced with a razor to produce rings. The width of each ring is approximately 3 mm. One ring is then press fitted onto each caplet such that the ring, or band, is located approximately at the midpoint of the length of the caplet. This step completes the fabrication procedure of the 625 mg banded caplet.

What is claimed is:

1. An oral dosage form comprising:
a compound of 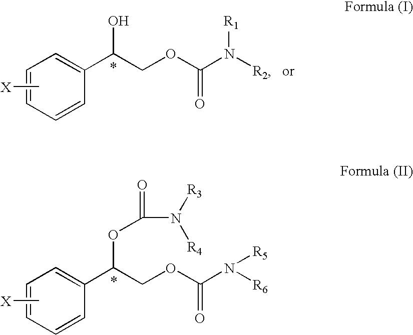
or pharmaceutically acceptable forms thereof, wherein phenyl is substituted at X with one to five halogen atoms selected from the group consisting of fluorine, chlorine, bromine and iodine, and R₁, R₂, R₃, R₄, R₅ and R₆ are independently selected from the group consisting of hydrogen and C₁-C₄ alkyl, wherein C₁-C₄ alkyl is optionally substituted with phenyl and, wherein phenyl is optionally substituted with substituents independently selected from the group consisting of halogen, C₁-C₄ alkyl, C₁-C₄ alkoxy, amino, nitro and cyano; and
an oral sustained release dosing structure.

2. The oral dosage form of claim 1, wherein the compound of Formula (I) or Formula (II) comprises an enantiomer selected from Formula (Ia) or Formula (IIa), a racemic mixture of Formula (Ia) or Formula (IIa), or an enantiomeric mixture of Formula (Ia) or Formula (IIa) wherein one enantiomer predominates: 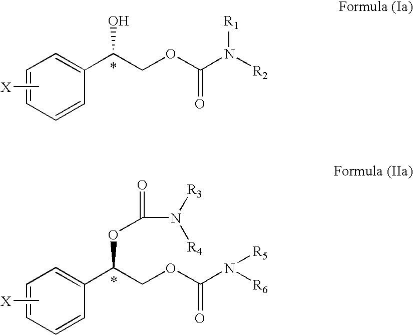
or pharmaceutically acceptable forms thereof, wherein phenyl is substituted at X with one to five halogen atoms selected from the group consisting of fluorine, chlorine, bromine and iodine, and R₁, R₂, R₃, R₄, R₅ and R₆ are independently selected from the group consisting of hydrogen and C₁-C₄ alkyl, wherein C₁-C₄ alkyl is optionally substituted with phenyl and, wherein phenyl is optionally substituted with substituents independently selected from the group consisting of halogen, C₁-C₄ alkyl, C₁-C₄ alkoxy, amino, nitro and cyano.

3. The oral dosage form of claim 2, wherein X is chlorine and, wherein X is substituted at the ortho position of the phenyl ring.

4. The oral dosage form of claim 2, wherein an enantiomer is selected from Formula (Ia) or Formula (IIa) or enantiomeric mixture thereof wherein one enantiomer predominates and, wherein R₁, R₂, R₃, R₄, R₅ and R₆ are hydrogen.

5. The oral dosage form of claim 4, wherein one enantiomer selected from Formula (Ia) or Formula (IIa) predominates and said enantiomer predominates to the extent of about 90% or greater.

6. The oral dosage form of claim 5, wherein one enantiomer selected from Formula (Ia) or Formula (IIa) predominates and said enantiomer predominates to the extent of about 98% or greater.

7. The oral dosage form of claim 1, wherein the compound of Formula (I) or Formula (II) comprises an enantiomer selected from Formula (Ib) or Formula (IIb), a racemic mixture of Formula (Ia) or Formula (IIa), or an enantiomeric mixture of Formula (Ib) or Formula (IIb) wherein one enantiomer predominates:

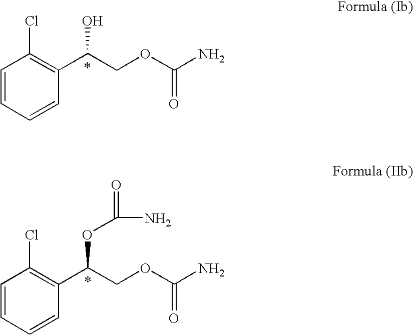

8. A method comprising:
comprising orally administering the oral dosage form of claim 1 to a patient.

9. A method comprising:
comprising orally administering the oral dosage form of claim 2 to a patient.

10. A method comprising:
comprising orally administering the oral dosage form of claim 7 to a patient.

11. An oral dosage form comprising:
a dose D of a compound of 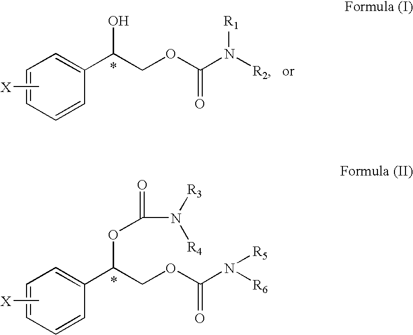
or pharmaceutically acceptable forms thereof, wherein phenyl is substituted at X with one to five halogen atoms selected from the group consisting of fluorine, chlorine, bromine and iodine, and R₁, R₂, R₃, R₄, R₅ and R₆ are independently selected from the group consisting of hydrogen and C₁-C₄ alkyl, wherein C₁-C₄ alkyl is optionally substituted with phenyl and, wherein phenyl is optionally substituted with substituents independently selected from the group consisting of halogen, C₁-C₄ alkyl, C₁-C₄ alkoxy, amino, nitro and cyano; and
an oral sustained release dosing structure adapted to sustainably release the compound of Formula (I) or Formula (II) at rates that provide mean, single dose, maximum plasma concentration Cₘₐₓ of the compound of Formula (I) or Formula (II), which satisfy the relationship:
about 5 ng/mL/mg≦Cmax/D≦about 16 ng/mL/mg.

12. The oral dosage form of claim 11, wherein the mean, single dose, maximum plasma concentration Cₘₐₓ of the compound of Formula (I) or Formula (II), satisfies the relationship:
about 7 ng/mL/mg≦Cmax/D≦about 14 ng/mL/mg.

13. The oral dosage form of claim 11, wherein the compound of Formula (I) or Formula (II) comprises an enantiomer selected from Formula (Ia) or Formula (IIa), a racemic mixture of Formula (Ia) or Formula (IIa), or an enantiomeric mixture of Formula (Ia) or Formula (IIa) wherein one enantiomer predominates: 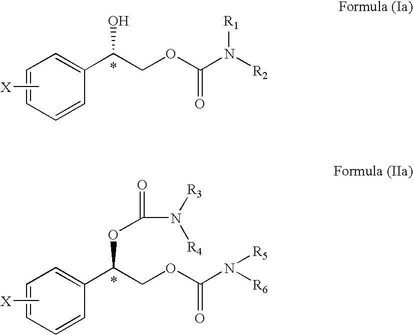
or pharmaceutically acceptable forms thereof, wherein phenyl is substituted at X with one to five halogen atoms selected from the group consisting of fluorine, chlorine, bromine and iodine, and R₁, R₂, R₃, R₄, R₅ and R₆ are independently selected from the group consisting of hydrogen and C₁-C₄ alkyl, wherein C₁-C₄ alkyl is optionally substituted with phenyl and, wherein phenyl is optionally substituted with substituents independently selected from the group consisting of halogen, C₁-C₄ alkyl, C₁-C₄ alkoxy, amino, nitro and cyano.

14. The oral dosage form of claim 13, wherein X is chlorine and, wherein X is substituted at the ortho position of the phenyl ring.

15. The oral dosage form of claim 13, wherein an-enantiomer is selected from Formula (Ia) or Formula (IIa) or enantiomeric mixture thereof wherein one enantiomer predominates and, wherein R₁, R₂, R₃, R₄, R₅ and R₆ are hydrogen.

16. The oral dosage form of claim 15, wherein one enantiomer selected from Formula (Ia) or Formula (IIa) predominates and said enantiomer predominates to the extent of about 90% or greater.

17. The oral dosage form of claim 16, wherein one enantiomer selected from Formula (Ia) or Formula (IIa) predominates and said enantiomer predominates to the extent of about 98% or greater.

18. The oral dosage form of claim 11, wherein the compound of Formula (I) or Formula (II) comprises an enantiomer selected from Formula (Ib) or Formula (IIb), a racemic mixture of Formula (Ia) or Formula (IIa), or an enantiomeric mixture of Formula (Ib) or Formula (IIb) wherein one enantiomer predominates:

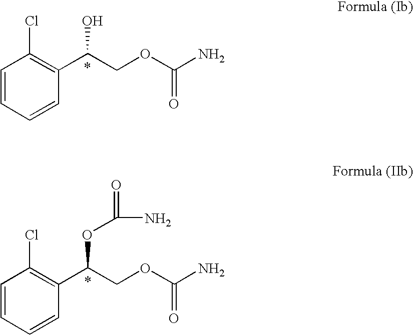

19. A method comprising:
comprising orally administering the oral dosage form of claim 11 to a patient.

20. A method comprising:
comprising orally administering the oral dosage form of claim 12 to a patient.

21. A method comprising:
comprising orally administering the oral dosage form of claim 13 to a patient.

22. A method comprising:
comprising orally administering the oral dosage form of claim 18 to a patient.

23. An oral dosage form comprising:
a dose D of a compound of 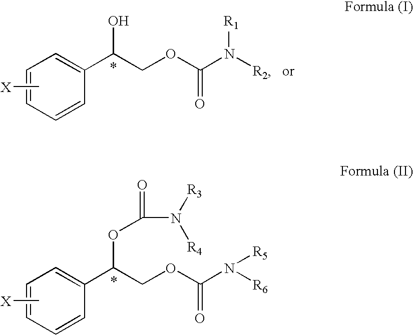
or pharmaceutically acceptable forms thereof, wherein phenyl is substituted at X with one to five halogen atoms selected from the group consisting of fluorine, chlorine, bromine and iodine, and R₁, R₂, R₃, R₄, R₅ and R₆ are independently selected from the group consisting of hydrogen and C₁-C₄ alkyl, wherein C₁-C₄ alkyl is optionally substituted with phenyl and, wherein phenyl is optionally substituted with substituents independently selected from the group consisting of halogen, C₁-C₄ alkyl, C₁-C₄ alkoxy, amino, nitro and cyano; and
an oral sustained release dosing structure adapted to sustainably release the compounds of Formula (I) or Formula (II) at rates that provide a mean, single dose, area under a plasma concentration-time curve AUC_{inf} of the compound of Formula (I) or of Formula (II) which satisfies the relationships:
about 150 ng hr/mL/mg≦AUC_{inf}/D≦about 425 ng hr/mL/mg.

24. The oral dosage form of claim 23, wherein the mean, single dose, area under a plasma concentration-time curve AUC_{inf} of the compound of Formula (I) or of Formula (II) satisfies the relationships:
about 220 ng hr/mL/mg≦AUC_{inf}/D≦about 360 ng hr/mL/mg.

25. The oral dosage form of claim 23, wherein the compound of Formula (I) or Formula (II) comprises an enantiomer selected from Formula (Ia) or Formula (IIa), a racemic mixture of Formula (Ia) or Formula (IIa), or an enantiomeric mixture of Formula (Ia) or Formula (IIa) wherein one enantiomer predominates: 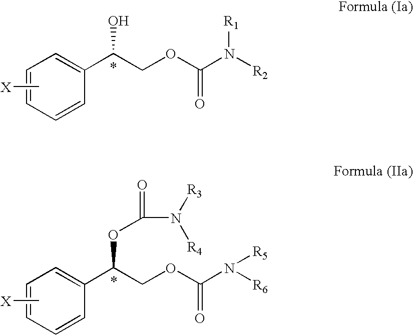
or pharmaceutically acceptable forms thereof, wherein phenyl is substituted at X with one to five halogen atoms selected from the group consisting of fluorine, chlorine, bromine and iodine, and R₁, R₂, R₃, R₄, R₅ and R₆ are independently selected from the group consisting of hydrogen and C₁-C₄ alkyl, wherein C₁-C₄ alkyl is optionally substituted with phenyl and, wherein phenyl is optionally substituted with substituents independently selected from the group consisting of halogen, C₁-C₄ alkyl, C₁-C₄ alkoxy, amino, nitro and cyano.

26. The oral dosage form of claim 25, wherein X is chlorine and, wherein X is substituted at the ortho position of the phenyl ring.

27. The oral dosage form of claim 24, wherein an enantiomer is selected from Formula (Ia) or Formula (IIa) or enantiomeric mixture thereof wherein one enantiomer predominates and, wherein R₁, R₂, R₃, R₄, R₅ and R₆ are hydrogen.

28. The oral dosage form of claim 27, wherein one enantiomer selected from Formula (Ia) or Formula (IIa) predominates and said enantiomer predominates to the extent of about 90% or greater.

29. The oral dosage form of claim 28, wherein one enantiomer selected from Formula (Ia) or Formula (IIa) predominates and said enantiomer predominates to the extent of about 98% or greater.

30. The oral dosage form of claim 23, wherein the compound of Formula (I) or Formula (II) comprises an enantiomer selected from Formula (Ib) or Formula (IIb), a racemic mixture of Formula (Ia) or Formula (IIa), or an enantiomeric mixture of Formula (Ib) or Formula (IIb) wherein one enantiomer predominates:

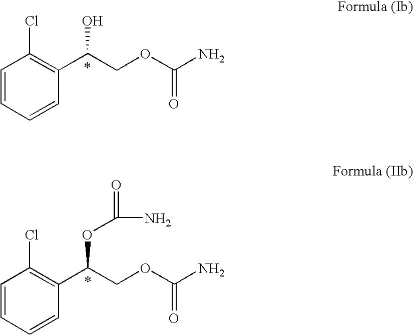

31. A method comprising:
comprising orally administering the oral dosage form of claim 23 to a patient.

32. A method comprising:
comprising orally administering the oral dosage form of claim 24 to a patient.

33. A method comprising:
comprising orally administering the oral dosage form of claim 25 to a patient.

34. A method comprising:
comprising orally administering the oral dosage form of claim 30 to a patient.

35. An oral dosage form comprising:
a dose D of a compound of 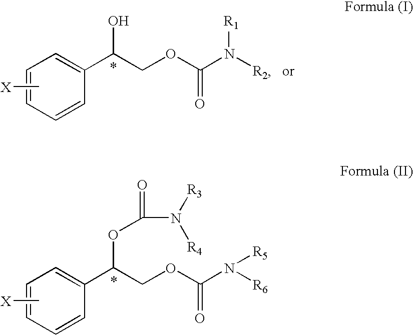
or pharmaceutically acceptable forms thereof, wherein phenyl is substituted at X with one to five halogen atoms selected from the group consisting of fluorine, chlorine, bromine and iodine, and R₁, R₂, R₃, R₄, R₅ and R₆ are independently selected from the group consisting of hydrogen and C₁-C₄ alkyl, wherein C₁-C₄ alkyl is optionally substituted with phenyl and, wherein phenyl is optionally substituted with substituents independently selected from the group consisting of halogen, C₁-C₄ alkyl, C₁-C₄ alkoxy, amino, nitro and cyano; and
an oral sustained release dosing structure adapted to sustainably release the compound of Formula (I) or Formula (II) at rates that provide (a) a mean, single dose, area under a plasma concentration-time curve AUC_{inf} of the compound of Formula (I) or Formula (II) and (b) a mean, single dose, maximum plasma concentration Cₘₐₓ of the compound of Formula (I) or Formula (II), which satisfy the relationships:
about 150 ng hr/mL/mg≦AUC_{inf}/D≦about 425 ng hr/mL/mg,
and
about 5 ng/mL/mg≦Cmax/D≦about 16 ng/mL/mg.

36. The oral dosage form of claim 35, wherein the mean, single dose, area under a plasma concentration-time curve AUC_{inf} of the compound of Formula (I) or Formula (II) and the mean, single dose, maximum plasma concentration Cₘₐₓ of the compound of Formula (I) or Formula (II), satisfy the relationships:
about 220 ng hr/mL/mg≦AUC_{inf}/D≦about 360 ng hr/mL/mg,
and
about 7 ng/mL/mg≦Cmax/D≦about 14 ng/mL/mg.

37. The oral dosage form of claim 35, wherein the compound of Formula (I) or Formula (II) comprises an enantiomer selected from Formula (Ia) or Formula (IIa), a racemic mixture of Formula (Ia) or Formula (IIa), or an enantiomeric mixture of Formula (Ia) or Formula (IIa) wherein one enantiomer predominates: 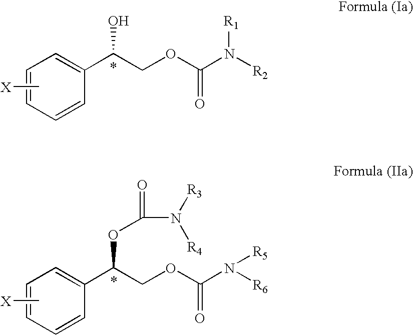
or pharmaceutically acceptable forms thereof, wherein phenyl is substituted at X with one to five halogen atoms selected from the group consisting of fluorine, chlorine, bromine and iodine, and R₁, R₂, R₃, R₄, R₅ and R₆ are independently selected from the group consisting of hydrogen and C₁-C₄ alkyl, wherein C₁-C₄ alkyl is optionally substituted with phenyl and, wherein phenyl is optionally substituted with substituents independently selected from the group consisting of halogen, C₁-C₄ alkyl, C₁-C₄ alkoxy, amino, nitro and cyano.

38. The oral dosage form of claim 37, wherein X is chlorine and, wherein X is substituted at the ortho position of the phenyl ring.

39. The oral dosage form of claim 37, wherein an enantiomer is selected from Formula (Ia) or Formula (IIa) or enantiomeric mixture thereof wherein one enantiomer predominates and, wherein R₁, R₂, R₃, R₄, R₅ and R₆ are hydrogen.

40. The oral dosage form of claim 39, wherein one enantiomer selected from Formula (Ia) or Formula (IIa) predominates and said enantiomer predominates to the extent of about 90% or greater.

41. The oral dosage form of claim 40, wherein one enantiomer selected from Formula (Ia) or Formula (IIa) predominates and said enantiomer predominates to the extent of about 98% or greater.

42. The oral dosage form of claim 35, wherein the compound of Formula (I) or Formula (II) comprises an enantiomer selected from Formula (Ib) or Formula (IIb), a racemic mixture of Formula (Ia) or Formula (IIa), or an enantiomeric mixture of Formula (Ib) or Formula (IIb) wherein one enantiomer predominates:

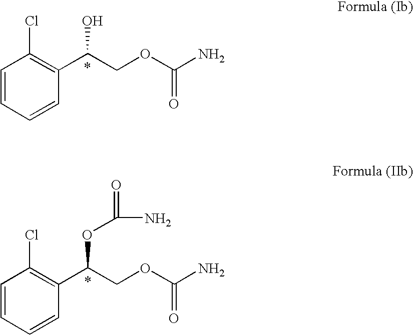

43. A method comprising:
comprising orally administering the oral dosage form of claim 35 to a patient.

44. A method comprising:
comprising orally administering the oral dosage form of claim 36 to a patient.

45. A method comprising:
comprising orally administering the oral dosage form of claim 37 to a patient.

46. A method comprising:
comprising orally administering the oral dosage form of claim 42 to a patient.

47. An oral dosage form comprising:
a dose D of a compound of 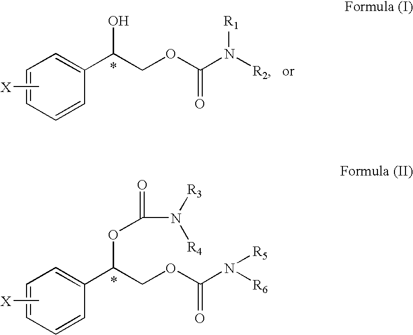
or pharmaceutically acceptable forms thereof, wherein phenyl is substituted at X with one to five halogen atoms selected from the group consisting of fluorine, chlorine, bromine and iodine, and R₁, R₂, R₃, R₄, R₅ and R₆ are independently selected from the group consisting of hydrogen and C₁-C₄ alkyl, wherein C₁-C₄ alkyl is optionally substituted with phenyl and, wherein phenyl is optionally substituted with substituents independently selected from the group consisting of halogen, C₁-C₄ alkyl, C₁-C₄ alkoxy, amino, nitro and cyano; and
an oral sustained release dosing structure adapted to sustainably release the compound of Formula (I) or Formula (II) at rates that provide (a) a mean, single dose, areas under a plasma concentration-time curve AUC of the compound of Formula (I) or Formula (II) for 0-3 hours AUC₀₋₃, 3-6 hours AUC₃₋₆, 6-9 hours AUC₆₋₉, 9-12 hours AUC₉₋₁₂, and 0-12 hours AUC₀₋₁₂ which satisfy the relationships:
AUC₀₋₃/AUC₀₋₁₂≧0.18,
AUC₃₋₆/AUC₀₋₁₂≧0.18,
AUC₆₋₉/AUC₀₋₁₂≧0.18, and
AUC₉₋₁₂/AUC₀₋₁₂≧0.18.

48. An oral dosage form comprising:
a dose D of a compound of 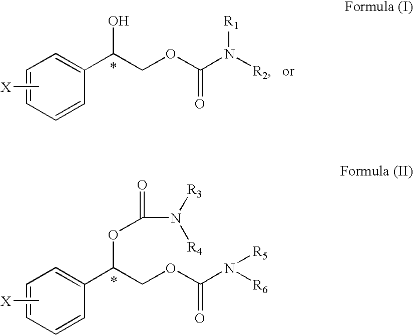
or pharmaceutically acceptable forms thereof, wherein phenyl is substituted at X with one to five halogen atoms selected from the group consisting of fluorine, chlorine, bromine and iodine, and R₁, R₂, R₃, R₄, R₅ and R₆ are independently selected from the group consisting of hydrogen and C₁-C₄ alkyl, wherein C₁-C₄ alkyl is optionally substituted with phenyl and, wherein phenyl is optionally substituted with substituents independently selected from the group consisting of halogen, C₁-C₄ alkyl, C₁-C₄ alkoxy, amino, nitro and cyano; and
an oral sustained release dosing structure adapted to sustainably release the compound of Formula (I) or Formula (II) at rates that provide (a) a mean, single dose, areas under a plasma concentration-time curve AUC of the compound of Formula (I) or Formula (II) for 0-6 hours AUC₀₋₆, 6-12 hours AUC₆₋₁₂, and 0-12:hours AUC₀₋₁₂ which satisfy the relationships:
AUC₀₋₆/AUC₀₋₁₂≧0.36,
AUC₆₋₁₂/AUC₀₋₁₂≧0.36.

49. An oral dosage form comprising:
a dose D of a compound of 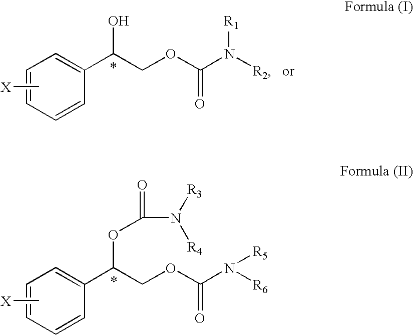
or pharmaceutically acceptable forms thereof, wherein phenyl is substituted at X with one to five halogen atoms selected from the group consisting of fluorine, chlorine, bromine and iodine, and R₁, R₂, R₃, R₄, R₅ and R₆ are independently selected from the group consisting of hydrogen and C₁-C₄ alkyl, wherein C₁-C₄ alkyl is optionally substituted with phenyl and, wherein phenyl is optionally substituted with substituents independently selected from the group consisting of halogen, C₁-C₄ alkyl, C₁-C₄ alkoxy, amino, nitro and cyano; and
an oral sustained release dosing structure adapted to sustainably release the compound of Formula (I) or Formula (II) at rates that provide (a) a mean, single dose, areas under a plasma concentration-time curve AUC of the compound of Formula (I) or Formula (II) for 0-3 hours AUC₀₋₃, 3-6 hours AUC₃₋₆, 6-9 hours AUC₆-9, and 9-12 hours AUC₉₋₁₂, wherein the ratios of each of:
AUC₀₋₃/AUC₃₋₆,
AUC₃₋₆/AUC₆₋₉, and
AUC₆₋₉/AUC₉₋₁₂
are between about 0.7 to about 1.33.

50. An oral dosage form comprising:
a dose D of a compound of 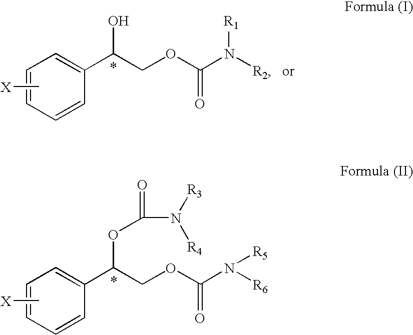
or pharmaceutically acceptable forms thereof, wherein phenyl is substituted at X with one to five halogen atoms selected from the group consisting of fluorine, chlorine, bromine and iodine, and R₁, R₂, R₃, R₄, R₅ and R₆ are independently selected from the group consisting of hydrogen and C₁-C₄ alkyl, wherein C₁-C₄ alkyl is optionally substituted with phenyl and, wherein phenyl is optionally substituted with substituents independently selected from the group consisting of halogen, C₁-C₄ alkyl, C₁-C₄ alkoxy, amino, nitro and cyano; and
an oral sustained release dosing structure adapted to sustainably release the compound of Formula (I) or Formula (II) at rates that provide (a) a mean, single dose, areas under a plasma concentration-time curve AUC of the compound of Formula (I) or Formula (II) for 0-6 hours AUC₀₋₆, 6-12 hours AUC₆₋₁₂, 12-18 hours AUC₁₂₋₁₈, 18-24 hours AUC₁₈₋₂₄, and 0-24 hours AUC₀₋₂₄ satisfy the relationships:
AUC₀₋₆/AUC₀₋₂₄≧0.18,
AUC₆₋₁₂/AUC₀₋₂₄≧0.18,
AUC₁₂₋₁₈/AUC₀₋₂₄≧0.18, and
AUC₁₈₋₂₄/AUC₀₋₂₄≧0.18.

51. An oral dosage form comprising:
a dose D of a compound of 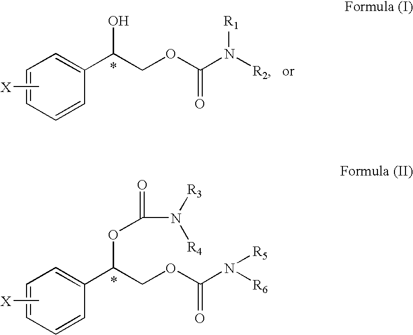
or pharmaceutically acceptable forms thereof, wherein phenyl is substituted at X with one to five halogen atoms selected from the group consisting of fluorine, chlorine, bromine and iodine, and R₁, R₂, R₃, R₄, R₅ and R₆ are independently selected from the group consisting of hydrogen and C₁-C₄ alkyl, wherein C₁-C₄ alkyl is optionally substituted with phenyl and, wherein phenyl is optionally substituted with substituents independently selected from the group consisting of halogen, C₁-C₄ alkyl, C₁-C₄ alkoxy, amino, nitro and cyano; and
an oral sustained release dosing structure adapted to sustainably release the compound of Formula (I) or Formula (II) at rates that provide (a) a mean, single dose, areas under a plasma concentration-time curve AUC of the compound of Formula (I) or Formula (II) for 0-12 hours AUC₀₋₁₂, 12-24 hours AUC₁₂₋₂₄, and 0-24 hours AUC₀₋₂₄ which satisfy the relationships:
AUC₀₋₁₂/AUC₀₋₂₄≧0.36,
AUC₁₂₋₂₄/AUC₀₋₂₄≧0.36.

52. An oral dosage form comprising:
a dose D of a compound of 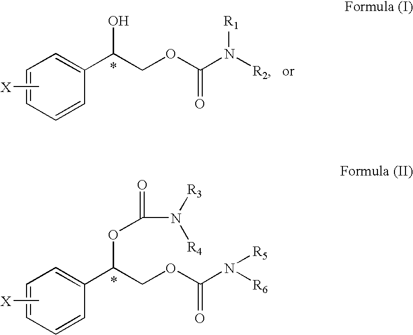
or pharmaceutically acceptable forms thereof, wherein phenyl is substituted at X with one to five halogen atoms selected from the group consisting of fluorine, chlorine, bromine and iodine, and R₁, R₂, R₃, R₄, R₅ and R₆ are independently selected from the group consisting of hydrogen and C₁-C₄ alkyl, wherein C₁-C₄ alkyl is optionally substituted with phenyl and, wherein phenyl is optionally substituted with substituents independently selected from the group consisting of halogen, C₁-C₄ alkyl, C₁-C₄ alkoxy, amino, nitro and cyano; and
an oral sustained release dosing structure adapted to sustainably release the compound of Formula (I) or Formula (II) at rates that provide (a) a mean, single dose, areas under a plasma concentration-time curve AUC of the compound of Formula (I) or Formula (II) for 0-6 hours AUC₀₋₆, 6-12 hours AUC₆₋₁₂, 12-18 hours AUC₁₂₋₁₈, and 18-24 hours AUC₁₈₋₂₄, wherein the ratios:
AUC₀₋₆/AUC₆₋₁₂
AUC₆₋₁₂/AUC₁₂₋₁₈, and
AUC₁₂₋₁₈/AUC₁₈₋₂₄
are between about 0.7 to about 1.33.

53. An oral dosage form comprising:
a dose D of a compound of 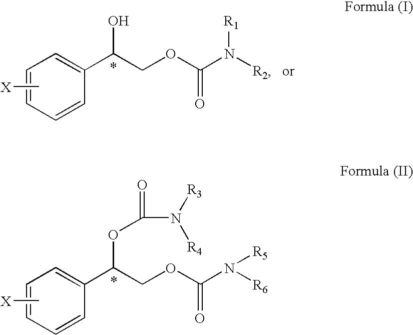
or pharmaceutically acceptable forms thereof, wherein phenyl is substituted at X with one to five halogen atoms selected from the group consisting of fluorine, chlorine, bromine and iodine, and R₁, R₂, R₃, R₄, R₅ and R₆ are independently selected from the group consisting of hydrogen and C₁-C₄ alkyl, wherein C₁-C₄ alkyl is optionally substituted with phenyl and, wherein phenyl is optionally substituted with substituents independently selected from the group consisting of halogen, C₁-C₄ alkyl, C₁-C₄ alkoxy, amino, nitro and cyano; and
an oral sustained release dosing structure adapted to sustainably release the compound of Formula (I) or Formula (II) at rates that provide (a) a mean, single dose, areas under a plasma concentration-time curve AUC of the compound of Formula (I) or Formula (II) for 0-3 hours AUC₀₋₃, 3-6 hours AUC₃₋₆, 6-9 hours AUC₆₋₉, 9-12 hours AUC₉₋₁₂, and 0-12 hours AUC₀₋₁₂ which satisfy the relationships:
about 0.02≧AUC₀₋₃/AUC₀₋₁₂≦about 0.15,
about 0.15≧AUC₃₋₆/AUC₀₋₁₂≦about 0.30,
about 0.20≧AUC₆₋₉/AUC₀₋₁₂≦about 0.40, and
about 0.30≧AUC₉₋₁₂/AUC₀₋₁₂—about 0.50.

54. An oral dosage form comprising:
a dose D of a compound of 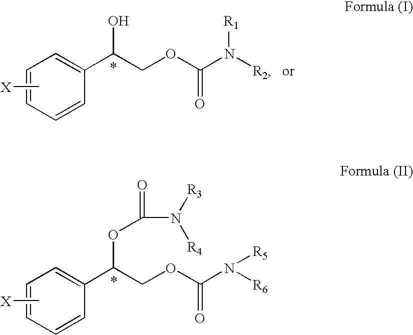
or pharmaceutically acceptable forms thereof, wherein phenyl is substituted at X with one to five halogen atoms selected from the group consisting of fluorine, chlorine, bromine and iodine, and R₁, R₂, R₃, R₄, R₅ and R₆ are independently selected from the group consisting of hydrogen and C₁-C₄ alkyl, wherein C₁-C₄ alkyl is optionally substituted with phenyl and, wherein phenyl is optionally substituted with substituents independently selected from the group consisting of halogen, C₁-C₄ alkyl, C₁-C₄ alkoxy, amino, nitro and cyano; and
an oral sustained release dosing structure adapted to sustainably release the compound of Formula (I) or Formula (II) at rates that provide (a) a mean, single dose, areas under a plasma concentration-time curve AUC of the compound of Formula (I) or Formula (II) for 0-6 hours AUC₀₋₆, 6-12 hours AUC₆₋₁₂, and 0-12 hours AUC₀₋₁₂ which satisfy the relationships:
about 0.1≧AUC₀₋₆/AUC₀₋₁₂≦about 0.5, and
about 0.5≧AUC₆₋₁₂/AUC₀₋₁₂≦about 0.9.

55. An oral dosage form comprising:
a dose D of a compound of 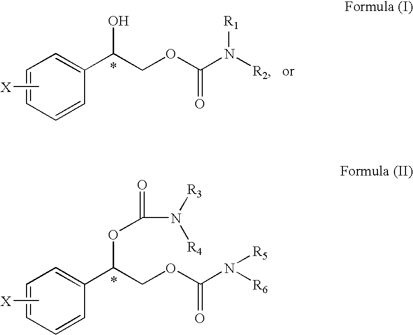
or pharmaceutically acceptable forms thereof, wherein phenyl is substituted at X with one to five halogen atoms selected from the group consisting of fluorine, chlorine, bromine and iodine, and R₁, R₂, R₃, R₄, R₅ and R₆ are independently selected from the group consisting of hydrogen and C₁-C₄ alkyl, wherein C₁-C₄ alkyl is optionally substituted with phenyl and, wherein phenyl is optionally substituted with substituents independently selected from the group consisting of halogen, C₁-C₄ alkyl, C₁-C₄ alkoxy, amino, nitro and cyano; and
an oral sustained release dosing structure adapted to sustainably release the compound of Formula (I) or Formula (II) at rates that provide (a) a mean, single dose, areas under a plasma concentration-time curve AUC of the compound of Formula (I) or Formula (II) for 0-6 hours AUC₀₋₆, 6-12 hours AUC₆₋₁₂, 12-18 hours AUC₁₂₋₁₈, 18-24 hours AUC₁₈₋₂₄, and 0-24 hours AUC₀₋₂₄ satisfy the relationships:
about 0.02≧AUC₀₋₆/AUC₀₋₂₄≦about 0.20,
about 0.15≧AUC₆₋₁₂/AUC₀₋₂₄≦about 0.30,
about 0.20 AUC₁₂₋₁₈/AUC₀₋₂₄≦about 0.40 and
about 0.25≧AUC₁₈₋₂₄/AUC₀₋₂₄≦about 0.50.

56. An oral dosage form comprising:
a dose D of a compound of 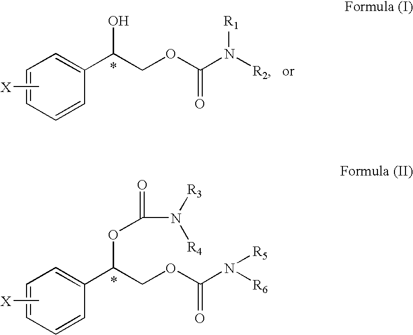
or pharmaceutically acceptable forms thereof, wherein phenyl is substituted at X with one to five halogen atoms selected from the group consisting of fluorine, chlorine, bromine and iodine, and R₁, R₂, R₃, R₄, R₅ and R₆ are independently selected from the group consisting of hydrogen and C₁-C₄ alkyl, wherein C₁-C₄ alkyl is optionally substituted with phenyl and, wherein phenyl is optionally substituted with substituents independently selected from the group consisting of halogen, C₁-C₄ alkyl, C₁-C₄ alkoxy, amino, nitro and cyano; and
an oral sustained release dosing structure adapted to sustainably release the compound of Formula (I) or Formula (II) at rates that provide (a) a mean, single dose, areas under a plasma concentration-time curve AUC of the compound of Formula (I) or Formula (II) for 0-12 hours AUC₀₋₁₂, 12-24 hours AUC₁₂₋₂₄, and 0-24 hours AUC₀₋₂₄ which satisfy the relationships:
about 0.1≦AUC₀₋₁₂/AUC₀₋₂₄≦about 0.55, and
about 0.45≦AUC₁₂₋₂₄/AUC₀₋₂₄≦about 0.9.

57. An oral dosage form comprising:
a dose of a compound of 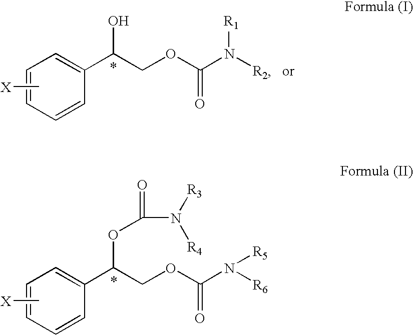
or pharmaceutically acceptable forms thereof, wherein phenyl is substituted at X with one to five halogen atoms selected from the group consisting of fluorine, chlorine, bromine and iodine, and R₁, R₂, R₃, R₄, R₅ and R₆ are independently selected from the group consisting of hydrogen and C₁-C₄ alkyl, wherein C₁-C₄ alkyl is optionally substituted with phenyl and, wherein phenyl is optionally substituted with substituents independently selected from the group consisting of halogen, C₁-C₄ alkyl, C₁-C₄ alkoxy, amino, nitro and cyano; and
an oral sustained release dosing structure adapted to sustainably release the compounds of Formula (I) or Formula (II) in vitro such that:
(a) the amount of the compounds of Formula (I) or Formula (II) released from the oral dosage form measured at 2 hours following initiation of release does not exceed about 10 wt % of the dose,
(b) the amount of the compounds of Formula (I) or Formula (II) released from the oral dosage form measured at 6 hours following initiation of release does not exceed about 40 wt % of the dose, or
(c) the amount of the compounds of Formula (I) or Formula (II) released from the oral dosage form measured at 12 hours following initiation of release does not exceed about 80 wt % of the dose;
wherein the amount released of the compounds of Formula (I) or Formula (II) is determined using a USP Type VII bath indexer in a constant temperature water bath at about 37° C.

58. An oral dosage form comprising:
a dose of a compound of 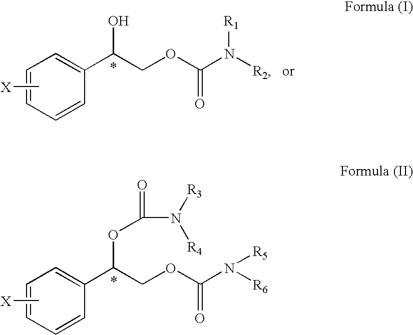
or pharmaceutically acceptable forms thereof, wherein phenyl is substituted at X with one to five halogen atoms selected from the group consisting of fluorine, chlorine, bromine and iodine, and R₁, R₂, R₃, R₄, R₅ and R₆ are independently selected from the group consisting of hydrogen and C₁-C₄ alkyl, wherein C₁-C₄ alkyl is optionally substituted with phenyl and, wherein phenyl is optionally substituted with substituents independently selected from the group consisting of halogen, C₁-C₄ alkyl, C₁-C₄ alkoxy, amino, nitro and cyano; and
an oral sustained release dosing structure adapted to sustainably release the compounds of Formula (I) or Formula (II) in vitro such that:
(a) the amount of the compounds of Formula (I) or Formula (II) released from the oral dosage form measured at 2 hours following initiation of release does not exceed about 10 wt % of the dose,
(b) the amount of the compounds of Formula (I) or Formula (II) released from the oral dosage form measured at 4 hours following initiation of release does not exceed about 40 wt % of the dose, or
(c) the amount of the compounds of Formula (I) or Formula (II) released from the oral dosage form measured at 7 hours following initiation of release does not exceed about 80 wt % of the dose;
wherein the amount released of the compounds of Formula (I) or Formula (II) is determined using a USP Type VII bath indexer in a constant temperature water bath at about 37° C.
